SEQUENCE LISTING (66 sequences)

SEQ ID NO: 1 - protein, 513 aa, Bacillus subtilis
MTLEKFVDALPIPDTLKPVQQTTEKTYYEVTMEECAHQLHRDLPPTRLWGYNGLFPGPTIEVKRNENVYVKWMNNLPSEHFLPIDHTIHHSDSQHEEPEVKTVVHLHGGVTPDDSDGYPEAWFSKDFEQTGPYFKREVYHYPNQQRGAILWYHDHAMALTRLNVYAGLVGDYIIHDPKEKRLKLPSGEYDVPLLITDRTINEDGSLFYPSGPENPSPSLPKPSIVPAFCGDTILVNGKVWPYLEVEPRKYRFRVINASNTRTYNLSLDNGGEFIQIGSDGGLLPRSVKLNSFSLAPAERYDIIIDFTAYEGESIILANSEGCGGDANPETDANIMQFRVTKPLAQKDESRKPKYLASYPSVQNERIQNIRTLKLAGTQDEYGRPVLLLNNKRWHDPVTEAPKAGTTEIWSIVNPTQGTHPIHLHLVSFRVLDRRPFDIARYQERGELSYTGPAVPPPPSEKGWKDTIQAHAGEVLRIAVTFGPYSGRYVWHCHILEHEDYDMMRPMDITDPHK

SEQ ID NO: 2 - protein, 539 aa, Bacillus subtilis
MTLEKFVDALPIPDTLKPVQQSKEKTYYEVTMEECTHQLHRDLPPTRLWGYNGLFPGPTIEVKRNENVYVKWMNNLPSTHFLPIDHTIHHSDSQHEEPEVKTVVHLHGGVTPDDSDGYPEAWFSKDFEQTGPYFKREVYHYPNQQRGAILWYHDHAMALTRLNVYAGLVGAYIIHDPKEKRLKLPSEEYDVPLLITDRTINEDGSLFYPSGPENPSPSLPNPSIVPAFCGETILVNGKVWPYLEVEPRKYRFRVINASNTRTYNLSLDNGGEFIQIGSDGGLLPRSVKLTSFSLAPAERYDIIIDFTAYEGQSIILANSAGCGGDVNPETDANIMQFRVTKPLAQKDESRKPKYLASYPSVQNERIQNIRTLKLAGTQDEYGRPVLLLNNKRWHDPVTEAPKAGTTEIWSIINPTRGTHPIHLHLVSFRVIDRRPFDIAHYQESGALSYTGPAVPPPPSEKGWKDTIQAHAGEVLRIAATFGPYSGRYVWHCHILEHEDYDMMRPMDITDPHKSDPNSSSVDKLHRTRAPPPPPLRSGC

SEQ ID NO: 3 - protein, 513 aa, Artificial Sequence
  COT1 introduced with Q386 and P487/W488/F489
MTLEKFVDALPIPDTLKPVQQTTEKTYYEVTMEECAHQLHRDLPPTRLWGYNGLFPGPTIEVKRNENVYVKWMNNLPSEHFLPIDHTIHHSDSQHEEPEVKTVVHLHGGVTPDDSDGYPEAWFSKDFEQTGPYFKREVYHYPNQQRGAILWYHDHAMALTRLNVYAGLVGDYIIHDPKEKRLKLPSGEYDVPLLITDRTINEDGSLFYPSGPENPSPSLPKPSIVPAFCGDTILVNGKVWPYLEVEPRKYRFRVINASNTRTYNLSLDNGGEFIQIGSDGGLLPRSVKLNSFSLAPAERYDIIIDFTAYEGESIILANSEGCGGDANPETDANIMQFRVTKPLAQKDESRKPKYLASYPSVQNERIQNIRTLKLAGTQDEYGRPVQLLNNKRWHDPVTEAPKAGTTEIWSIVNPTQGTHPIHLHLVSFRVLDRRPFDIARYQERGELSYTGPAVPPPPSEKGWKDTIQAHAGEVLRIAVTFGPYSGPWFWHCHILEHEDYDMMRPMDITDPHK

SEQ ID NO: 4 - protein, 513 aa, Artificial Sequence
  COT1 introduced with Q386
MTLEKFVDALPIPDTLKPVQQTTEKTYYEVTMEECAHQLHRDLPPTRLWGYNGLFPGPTIEVKRNENVYVKWMNNLPSEHFLPIDHTIHHSDSQHEEPEVKTVVHLHGGVTPDDSDGYPEAWFSKDFEQTGPYFKREVYHYPNQQRGAILWYHDHAMALTRLNVYAGLVGDYIIHDPKEKRLKLPSGEYDVPLLITDRTINEDGSLFYPSGPENPSPSLPKPSIVPAFCGDTILVNGKVWPYLEVEPRKYRFRVINASNTRTYNLSLDNGGEFIQIGSDGGLLPRSVKLNSFSLAPAERYDIIIDFTAYEGESIILANSEGCGGDANPETDANIMQFRVTKPLAQKDESRKPKYLASYPSVQNERIQNIRTLKLAGTQDEYGRPVQLLNNKRWHDPVTEAPKAGTTEIWSIVNPTQGTHPIHLHLVSFRVLDRRPFDIARYQERGELSYTGPAVPPPPSEKGWKDTIQAHAGEVLRIAVTFGPYSGRYVWHCHILEHEDYDMMRPMDITDPHK

SEQ ID NO: 5 - protein, 513 aa, Artificial Sequence
  COT1 introduced with P487/W488/F489
MTLEKFVDALPIPDTLKPVQQTTEKTYYEVTMEECAHQLHRDLPPTRLWGYNGLFPGPTIEVKRNENVYVKWMNNLPSEHFLPIDHTIHHSDSQHEEPEVKTVVHLHGGVTPDDSDGYPEAWFSKDFEQTGPYFKREVYHYPNQQRGAILWYHDHAMALTRLNVYAGLVGDYIIHDPKEKRLKLPSGEYDVPLLITDRTINEDGSLFYPSGPENPSPSLPKPSIVPAFCGDTILVNGKVWPYLEVEPRKYRFRVINASNTRTYNLSLDNGGEFIQIGSDGGLLPRSVKLNSFSLAPAERYDIIIDFTAYEGESIILANSEGCGGDANPETDANIMQFRVTKPLAQKDESRKPKYLASYPSVQNERIQNIRTLKLAGTQDEYGRPVLLLNNKRWHDPVTEAPKAGTTEIWSIVNPTQGTHPIHLHLVSFRVLDRRPFDIARYQERGELSYTGPAVPPPPSEKGWKDTIQAHAGEVLRIAVTFGPYSGPWFWHCHILEHEDYDMMRPMDITDPHK

SEQ ID NO: 6 - protein, 451 aa, Artificial Sequence
  Consnesus sequence
MTLEKFVDALPIPDTLKPVSKTYYEVTMEHKLHRDLPPTRLWGYNGLFPGPTIEVRNEVYVKWMNNLPHFLPVDHTIHSDHDEPEVKTVVHLHGGTPDSDGYPEAWFTKDFQTGPYFREVYHYPNQRGALWYHDHAMALTRLNVYAGLAGYIIRDKEKRLLPSGEYDIPLLIDRTFNEDGSLFYPSPENPSPSLPPSIVPAFCGETILVNGKVWPYLEVEPRKYRFRILNASNTRTYLSLDNGGFIQIGSDGGLLRPVKLNSLSLAPAERDIIIDFSAYEGSIILNGCGDPETDANIMQFRVTKPLAKDSRIPKLAIPLRIIRLKLGTDEYGRPVLLLNNKRWHDPVTEPKLGTTEIWSIINTGTHPIHLHLVQFRVLDRRPFDIRYQEGELYTGPAVPPPSEKGWKDTVQAAGEVLRIIAFGPYSGRYVWHCHILEHEDYDMMRPMDIID

SEQ ID NO: 7 - DNA, 43 bp, Bacillus subtilis
cgccgtctcacatgtcttttaaaaaatttgtcgatgcattacc

SEQ ID NO: 8 - DNA, 42 bp, Bacillus subtilis
atagttttggacgccctatgccattattaaataacatggagt

SEQ ID NO: 9 - DNA, 41 bp, Bacillus subtilis
cgcggatccgatgatttctcttcttttttatttttccgttg

SEQ ID NO: 10 - DNA, 42 bp, Bacillus subtilis
actccatgttatttaataatggcatagggcgtccaaaactat

SEQ ID NO: 11 - protein, 513 aa, Bacillus spec.
  misc_feature (35)..(35): Xaa can be any naturally occurring amino acid
MTLEKFVDALPIPDTLKPVQQSKEKTYYEVTMEEXTHQLHRDLPPTRLWGYNGLFPGPTIEVKRNENVYVKWMNNLPSTHFLPIDHTIHHSDSQHEEPEVKTVVHLHGGVTPDDSDGYPEAWFSKDFEQTGPYFKREVYHYPNQQRGAILWYHDHAMALTRLNVYAGLVGAYIIHDPKEKRLKLPSDEYDVPLLITDRTINEDGSLFYPSAPENPSPSLPNPSIVPAFCGETILVNGKVWPYLEVEPRKYRFRVINASNTRTYNLSLDNGGDFIQIGSDGGLLPRSVKLNSFSLAPAERYDIIIDFTAYEGESIILANSAGCGGDVNPETDANIMQFRVTKPLAQKDESRKPKYLASYPSVQHERIQNIRTLKLAGTQDEYGRPVLLLNNKRWHDPVTETPKVGTTEIWSIINPTRGTHPIHLHLVSFRVLDRRPFDIARYQESGELSYTGPAVPPPPSEKGWKDTIQAHAGEVLRIAATFGPYSGRYVWHCHALEHEDYDMMRPMDITDPHK

SEQ ID NO: 12 - protein, 513 aa, Bacillus spec.
  misc_feature (35)..(35): Xaa can be any naturally occurring amino acid
MTLEKFVDALPIPDTLKPVQQSKEKTYYEVTMEEXTHQLHRDLPPTRLWGYNGLFPGPTIEVKRNENVYVKWMNNLPSTHFLPIDHTIHHSDSQHEEPEVKTVVHLHGGVTPDDSDGYPEAWFSKDFEQTGPYFKREVYHYPNQQRGAILWYHDHAMALTRLNVYAGLVGAYIIHDPKEKRLKLPSDEYDVPLLITDRTINEDGSLFYPSAPENPSPSLPNPSIVPAFCGETILVNGKVWPYLEVEPRKYRFRVINASNTRTYNLSLDNGGDFIQIGSDGGLLPRSVKLNSFSLAPAERYDIIIDFTAYEGESIILANSAGCGGDVNPETDANIMQFRVTKPLAQKDESRKPKYLASYPSVQHERIQNIRTLKLAGTQDEYGRPVLLLNNKRWHDPVTETPKVGTTEIWSIINPTRGTHPIHLHLVSFRVLDRRPFDIARYQESGELSYTGPAVPPPPSEKGWKDTIQAHAGEVLRIAATFGPYSGRYVWHCHILEHEDYDMMRPMDITDPHK

SEQ ID NO: 13 - protein, 511 aa, Bacillus spec.
MTLEKFVDALPIPDTLKPVQQSKEKTYYEVTMEECTHQLHRDLPPTRLWGYNGLFPGPTIEVKRNENVYVKWMNNLPSTHFLPIDHTIHHSDSQHEEPEVKTVVHLHGGVTPDDSDGYPEAWFSKDFEQTGPYFKREVYHYPNQQRGAILWYHDHAMALTRLNVYAGLVGAYIIHDPKEKRLKLPSDEYDVPLLITDRTINEDGSLFYPSAPENPSPSLPNPSIVPAFCGETILVNGKVWPYLEVEPRKYRFRVINASNTRTYNLSLDNGGDFIQIGSDGGLLPRSVKLNSFSLAPAERYDIIIDFTAYEGESIILANSAGCGGDVNPETDANIMQFRVTKPLAQKTKAESRSTSPHTLRYSMKDTNIRTLKLAGTQDEYGRPVLLLNNKRWHDPVTETPKVGTTEIWSIINRHAEHILIHLHLVSFRVLDRRPFDIARYQESGELSYTVRCPAAASEKGWKDTIQAHAGEVLRIAATFGPYSGRYVWHCHILEHEDYDMMRPMDITDPHK

SEQ ID NO: 14 - protein, 513 aa, Bacillus spec.
MTLEKFVDALPIPDTLKPVQQSKEKTYYEVTMEECTHQLHRDLPPTRLWGYNGLFPGPTIEVKRNENVYVKWMNNLPSTHFLPIDHTIHHSDSQHEESEVKTVVHLHGGVTPDDSDGYPEAWFSKDFEQTGPYFKREVYHYPNQQRGAILWYHDHAMALTRLNVYAGLVGAYIIHDPKEKRLKLPSDEYDVPLLITDRTINEDGSLFYPSAPENPSPSLPNPSIVPAFCGETILVNGKVWPYLEVEPRKYRFRVINASNTRTYNLSLDNGGEFIQVGSDGGLLPRSVKLNSFSLAPAERYDIIIDFTAYEGESIILANSAGCGGDVNPETDANIMQFRVTKPLAQKDESRKPEYLASYPSVQHERIQNIRTLKLAGTQDEYGRPVLLLNNKRWHDPVTGAPKVGTTEIWSIINPTRGTHPIHLHLVSFRVLDRRPFDIARYQESGELSYTGPAVPPPPSEEGWKDTIQAHAGEVLRIAATFGPYSGRYVWHCHILEHEDYDMMRPMDITDRHK

SEQ ID NO: 15 - protein, 513 aa, Bacillus spec.
MTLEKFVDALPIPDTLKPVQQSKEKTYYEVTMEECTHQLHRDLPPTRLWGYNGLFPGPTIEVKRNENVYVKWMNNLPSTRFLPIDHTIHHSDSQHEEPEVKTVVHLHGGVTPDDSDGYPEAWFSKDFEQTGPYFKREVYHYPNQQRGAILWYHDHAMALTRLNVYAGLVGAYIIHDPKEKRLKLPSDEYDVPLLITDRTINEDGSLFYPSAPENPSPSLPNPSIVPAFCGETILVNGKVWPYLEVEPRKYRFRVINASNTRTYNLSLDNGGEFIQIGSDGGLLPRSVKLNSFSLAPAERYDIIIDFTAYEGESIILANSAGCGGDVNPETDANIMQFRVTKPLAQKDESRKPKYLASYPSVQHERIQNIRTLKLAGTQGEYGRPVLLLNNKRWHDPVTEAPKVGTAEIWSIINPTRGTHPIHLHLVSFRVLDRRPFDIARYQESGELSYTGPAVPPPPSEKGWKDTIQAHAGEVLRIAATFGPYSGRYVWHCHILEHEDYDMMRPMDITDPHK

SEQ ID NO: 16 - protein, 513 aa, Bacillus spec.
MTLEKFVDALPIPDTLKPVQQSKDSTYYEVTMEECYHQLHRDLPPTRLWGYNGLFPGPTIKAKRNENVYVKWMNNLPSEHFLPIDHTIHHSDSQHAEPEVKTVVHLHGGVTPDDSDGYPEAWFSKDFEQTGPYFKREVYHYPNQQRGAILWYHDHAMALTRLNVYAGLIGAYIIHEPKEKRLKLPSGEYDVPLLITDRTINEDGSLFYPSGPENPSPSLPNPSIVPAFCGDTILVNGKAWPYMEVEPRKYRFRVINASNTRTYNLSLDNGGEFIQIGSDGGLLPRSVKLNSFSIAPAERFDILIDFAAFEGQSIILANSEGCGGDVNPETDANIMQFRVTKPLAQKDESRKPKYLASYPSVQHERIQNLRTLKLAGTQDQYGRPVLLLNNKRWHDPVTEAPKVGSTEIWSIINPTRGTHPIHLHLVSFRVLDRRPFDTARFEERGELAYTGPAVPPPPSEKGWKDTVQSHAGEVLRIAVTFGPYTGRYVWHCHILEHEDYDMMRPMDITDPHK

SEQ ID NO: 17 - protein, 515 aa, Bacillus spec.
MKMTLEKFVDALPIPDTLKPVQQSKEKTYYEVTMEECTHQLHRDLPPTRLWGYNGLFPGPTIEVKRNENVYVKWMNNLPSTHFLPIDHTIHHSDSQHEEPEVKTVVHLHGGVTPDDSDGYPEAWFSKDFEQTGPYFKREVYHYPNQQRGAILWYHDHAMALTRLNVYAGLVGAYIIHDPKEKRLKLPSDEYDVPLLITDRTINEDGSLFYPSAPENPSPSLPNPSIVPAFCGETILVNGKVWPYLEVEPRKYRFRVINASNTRTYNLSLDNGGDFIQIGSDGGLLPRSVKLNSFSLAPAERYDIIIDFTAYEGESIILANSAGCGGDVNPETDANIMQFRVTKPLAQKDESRKPKYLASYPSVQHERIQNIRTLKLAGTQDEYGRPVLLLNNKRWHDPVTETPKVGTTEIWSIINPTRGTHPIHLHLVSFRVLDRRPFDIARYQESGELSYTGPAVPPPPSEKGWKDTIQAHAGEVLRIAATFGPYSGRYVWHCHILEHEDYDMMRPMDITDPHK

SEQ ID NO: 18 - protein, 513 aa, Bacillus spec.
MTLEKFVDALPIPDTLKPVQQSKEKTYYEVTMEECTHQLHRDLPPTRLWGYNGLFPGPTIEVKRNENVYVKWMNNLPSTHFLPIDHTIHHSDSQHEEPEVKTVVHLHGGVTPDDSDGYPEAWFSKDFEQTGPYFKREVYHYPNQQRGAILWYHDHAMALTRLNVYAGLVGAYIIHDPKEKRLKLPSDEYDVPLLITDRTINEDGSLFYPSAPENPSPSLPNPSIVPAFCGETILVNGKVWPYLEVEPRKYRFRVINASNTRTYNLSLDNGGDFIQIGSDGGLLPRSVKLNSFSLAPAERYDIIIDFTAYEGESIILANSAGCGGDVNPETDANIMQFRVTKPLAQKDESRKPKYLASYPSVQHERIQNIRTLKLAGTQDEYGRPVLLLNNKRWHDPVTETPKVGTTEIWSIINPTRGTHPIHLHLVSFRVLDRRPFDIARYQESGELSYTGPAVPPPPSEKGWKDTIQAHAGEVLRIAATFGPYSGRYVWHCHILEHEDYDMMRPMDITDPHK

SEQ ID NO: 19 - protein, 513 aa, Bacillus spec.
MKLTKFVDELPIIQTLHPTEKSNHLTYYEVTMKEGWHKLHRDLPSTKLWGYNGQFPGPTIEVSEREMIHVKWMNHLPPKLMLPLDTSIHHLDQMPQTRTVTHVHGSVTKPDSDGYPEAWFTRGFQETGPDFSREVYEYPNNQRAATLWYHDHAMGITRLNVYAGLVGMYIIRGEEEKALQLPSGDYEIPLIICDRTLNPDGSLFYPSGPDEPIPGAPSPSIVPAYLGETILVNGKAWPYMDVEPRKYRFRLVNASNTRTYRISLNNDVPIYQIGSDGGLLRKSIPTRQFVIEPAERIDIVIDFSEYKGQVININNDLGEDADPDDQTNHIMQFKVYKPLKNKDISRIPKHLTHIPSLKQNSINTIRNLKLVGSEDKFGRPLLLLNNQLWHDPITEKPQLGDTEIWSIVNVTNFTHPIHLHLVQFQVLDRQPFDLEKYNEDGSIIYTDAPLSPAENEKSWKDTLAAPSAHVTRVIAKFEPFTGDYVWHCHILEHEDYDMMRPFTIVDKEEEETL

SEQ ID NO: 20 - protein, 512 aa, Bacillus spec.
MALEKFADELPIIETLQPQKKSDGSTYYEVTMKECFHKLHRDLPPTRLWGYNGLFPGPTIDVNKDENVYIKWMNDLPDKHFLPVDHTIHHSESGHQEPDVKTVVHLHGGATPPDSDGYPEAWFTKDFKETGPYFEREVYHYPNKQRGALLWYHDHAMAITRLNVYAGLAGMYIIRERKEKQLKLPSGEYDVPLMILDRTLNDDGSLSYPSGPANPSETLPDPSIVPFFCGNTILVNGKAWPYMEVEPRTYRFRILNASNTRTFSLSLNNGGKFIQIGSDGGLLPRSVKTQSISLAPAERYDVLIDFSAFDGEHIILTNGTGCGGDADPDTDANVMQFRVTKPLKGADTSRKPKYLSTMPNVTSKRIHNIRMLKLTNTQDKYGRPVLTLNNKRWHDPVTEAPKLGTTEIWSIINPMGGTHPIHLHLVSFQILDRRPFDLERYNKFGDIVYTGPAVPPPPSEKGWKDTVQAHSGEVIRIAATFAPYSGRYVWHCHILEHEDYDMMRPMDVTDKQ

SEQ ID NO: 21 - protein, 509 aa, Bacillus spec.
MNLEKFVDELPIPEVAEPVKKNPRQTYYEIAMEEVFLKVHRDLPPTKLWTYNGSLPGPTIHANRNEKVKVKWMNKLPLKHFLPVDHTIHEGHHDEPEVKTVVHLHGGVTPASSDGYPEAWFSRDFEATGPFFEREVYEYPNHQQACTLWYHDHAMALTRLNVYAGLAGFYLISDAFEKSLELPKDEYDIPLMIMDRTFQEDGALFYPSRPNNTPEDSDLPDPSIVPFFCGETILVNGKVWPYLEVEPRKYRFRILNASNTRTYELHLDNDATILQIGSDGGFLPRPVHHQSFTIAPAERFDVIIDFSAYENKTITLKNKAGCGQEVNPETDANIMQFKVTRPLKGRAPKTLRPIFKPLPPLRPSRADQERTLTLTGTQDKYGRPILLLDNHFWNDPVTENPRLGSVEVWSIVNPTRGTHPIHLHLVQFRVIDRRPFDTEVYQSTGDIVYTGPNEAPPLHEQGYKDTIQAHAGEVIRIIARFVPYSGRYVWHCHILEHEDYDMMRPMDII

SEQ ID NO: 22 - protein, 615 aa, Bacillus spec.
MSLPINPSDPATIPKFVDALQKPVTAKPKYCRGQKKHDYYELVMMEGEHRFHKHFPNTLIWGYNGLYPGPTIEATKDKTIYVKYKNQLPLQHFLPVDFTLHAANDSQEVRTVTHLHGANVDWESDGHPEAWYTRDYRHTGPKFNKEIHEYTNHQPGTTMWYHDHAMALTRLNVYAGLAGFYLLRDALEERSNLPCGEYEYPILIQDKSFNEDGSLFYPDEPPFPVTVHPSITPGFVGNTIVVNGKLWPYLDVEPRKYRFRFLNGSNRRGYVISLSNEQSMIQIGTDGGFLSTPQAIQSVELLPAERTDVIIDFSACEGQEITLLNTDTDFIDEHTNVIMQFRVSLPLKYEDTSEVPERLAVTMDLHPHHAHIERQLPLTATTDEFGRPMLLLNNRMYHDPATEKPSLDSIEIWSFINTTPFIHPIHLHLIQFKILERRPFDLDLYQNEGIVSFTGPPEEPRDYERGWKDTVKVDAGKITKIIMHWKDHVGNYMWHCHFLEHEDHDMMRPILVMKDVHAVQQPHAEIEPSSHQEVTTTTTSTPTTNTSTTHHNESPVPLLNTSQIPSTEEEEIKESLVEESVHIGNNVQHRPILLFPTKSTPAGTSRPRRRGHRRF

SEQ ID NO: 23 - protein, 523 aa, Bacillus spec.
MPLEKFVDPLPIPGILKPVGKLNGVDFYRVAMREFKQKLHRDLPETTVWGYEGTYPGPTFEVRKNVPIKVKWENNLPVNRHLLPVDRTVHGAQPPNPLVRTVVHLHGGNVPDESDGYPEAWFTNDFEQTGPFFTQKIYDYPNAQPATTLWYHDHALGITRLNVYAGLAGFYLIRDDCEDQLNLPKGNFEIPLVIQDRSFNPDGSLFYPRQPDPPPEGFPPGFPDPSIVPEFFGDTILVNGKVWPFLEVEPRKYRFRLLNGSNSRFYRMKLTSGQPFIQIGTDQGFLKKPVVTNEITLGPGERADVVLDFTAFTGQNIILTNDAPTPFPNGNPVDPNTDPVGQLMQFRVNLPLSGPDTSIVPSRLCSLPRLKEEMAGVVRDQTLVESTDEFGRLLLLLEGLRWSDPITDEPELGNIEIWRLINTTPDTHPIHLHLVRFLILDRQPFDVKRFQETDEIVFTGPAEPPDPTERGWKDTVRTNPGEITRIIIRFVSYTGVFPWHCHILEHEDNEMMRPYEVVAEEEE

SEQ ID NO: 24 - protein, 512 aa, Bacillus spec.
MALEKFADELPIIETLKPQKTSNGSTYYEVTMKECFHKLHRDLPPTRLWGYNGLFPGPTIDVNQDENVYIKWMNDLPDKHFLPVDHTIHHSEGGHQEPDVKTVVHLHGGATPPDSDGYPEAWFTRDFKEKGPYFEKEVYHYPNKQRGALLWYHDHAMAITRLNVYAGLAGMYIIRERKEKQLKLPAGEYDVPLMIMDRTLNDDGSLFYPSGPDNPSETLPNPSIVPFLCGNTILVNGKAWPYMEVEPRTYRFRILNASNTRTFSLSLNNGGRFIQIGSDGGLLPRSVKTQSISLAPAERYDVLIDFSAFDGEHIILTNGTGCGGDVNPDTDANVMQFRVTKPLKGEDTSRKPKYLSAMPDMTSKRIHNIRTLKLTNTQDKYGRPVLTLNNKRWHDPVTEAPRLGSTEIWSIINPTRGTHPIHLHLVSFQVLDRRPFDLERYNKFGDIVYTGPAVPPPPSEKGWKDTVQAHSGEVIRIAATFAPYSGRYVWHCHILEHEDYDMMRPMDVTEKQ

SEQ ID NO: 25 - protein, 513 aa, Bacillus spec.
MNLEKFADMLPIPEVLKPHQQTKESTYYEVTMKEFYQKLHRDLPPTRLWGYNGLFPGPTIEVNRNENVQIKWMNDLPDQHFLPIDHTIHHSEGHHQEPEVKTVVHLHGGATPYDSDGYPEAWFSKGFQETGPYFSREIYHYPNQQRGAILWYHDHAMALTRLNVYAGLAGVYIIHDPKEKRLKLPAGEYDVPLMIMDRTINEDGSLFYPSGPENPSPTLPTPSIVPAFCGDTILVNGKAWPYMEVEPRAYRFRIVNASNTRTYNLSLDNGGEFLQVGSDGGLLPRSVKLSSISLAPAERFDIIIDFAAFEGQSIVLANSEGCGGPANPESDANVMQFRVIKPLKEKDESRKPRFLTNLPPVTDEKIQNLRTLKLTGTQDEYGRPVLLLNNKRWSDPVTEAPKLGTSEIWSIINPTRGTHPIHLHLISFRVLDRRPFDTAKYAETGNVVFTGPAVPPPPSEKGWKDTVQSHAGEVIRIMAKFGPYSGRYVWHCHILEHEDYDMMRPMDVVDPNQ

SEQ ID NO: 26 - protein, 513 aa, Bacillus spec.
MTLEKFVDALPIPDTLKPVQQSKEKTYYEVTMEECTHQLHRDLPPTRLWGYNGLFPGPTIEVKRNENVYVKWMNNLPSTHFLPIDHTIHHSDSQHEEPEVKTVVHLHGGVTPDDSDGYPEAWFSKDFEQTGPYFKREVYHYPNQQRGAILWYHDHAMALTRLNVYAGLVGAYIIHDPKEKRLKLPSDEYDVPLLITDRTINEDGSLFYPSAPENPSPSLPNPSIVPAFCGETILVNGKVWPYLEVEPRKYRFRVINASNTRTYNLSLDNGGEFIQIGSDGGLLPRSVKLNSFSLAPAERYDIIIDFTAYEGESIILANSAGCGGDVNPETDANIMQFRVTKPLAQKDESRKPKYLASYPSVQHERIQNIRTLKLAGTQDEYGRPVLLLNNKRWHDPVTEAPKVGTTEIWSIINPTRGTHPIHLHLVSFRVLDRRPFDIARYQESGELSYTGPAVPPPPSEKGWKDTIQAHAGEVLRIAATFGPYSGRYVWHCHILEHEDYDMMRPMDITDPHK

SEQ ID NO: 27 - protein, 522 aa, Bacillus spec.
MSPNLEKFVDRLPLAEKIRPVREEGGIAYYEVTMEEFRQKLHRDLRPTRLWGYNRRFPGPLFDVPHGKKIRVKWTNHLPQRHFLPIDPTILDGMGTDFPEVRTVVHLHGGETKPDSDGYPEAWFTRDFNETGPAFKNEVYEYSNKQRPATLWYHDHAIGITRLNVYAGLAGMYIIRDQKEKVFHLPSGKYEIPLLLTDRTFNNDGSLFYPRQPQNPGPGTPDPSVVPFFLGDTILVNGKVWPYLEVEPRKYRFRIVNASNTRAYQLYLDSGQAFYQIGTDGGLLRRPVQVGNLALEPAERADLILDFSEYAGQTILLKNDLGPNADPADQTGDVMQFRVVLPVSGEDTSRIPPSLSSIPVPSSQNVSAIRHLKLTGATDSYGRPLLLLDKKRWMDPVTEMPRLGTTEIWSLANTTAFTHPIHIHLVQFQILDRRPFDLDLYNETGQIVYTGPATPPEPSERGFKDTVAAPGGQITRVMMRFSPYAGDYVWHCHILEHEDYDMMRPFQVIDPDLPESDSPLSD

SEQ ID NO: 28 - protein, 513 aa, Bacillus spec.
MKLEKFVDRLPIPQVLQPQSKSKEMTYYEVTMKEFQQQLHRDLPPTRLFGYNGVYPGPTFEVQKHEKVAVKWLNKLPDRHFLPVDHTLHDDGHHEHEVKTVVHLHGGCTPADSDGYPEAWYTKDFHAKGPFFEREVYEYPNEQDATALWYHDHAMAITRLNVYAGLVGLYFIRDREERSLNLPKGEYEIPLLIQDKSFHEDGSLFYPRQPDNPSPDLPDPSIVPAFCGDTILVNGKVWPFAELEPRKYRFRILNASNTRIFELYFDHDITCHQIGTDGGLLQHPVKVNELVIAPAERCDIIVDFSRAEGKTVTLKKRIGCGGQDADPDTDADIMQFRISKPLKQKDTSSLPRILRKRPFYRRHKINALRNLSLGAAVDQYGRPVLLLNNTKWHEPVTETPALGSTEIWSIINAGRAIHPIHLHLVQFMILDHRPFDIERYQENGELVFTGPAVPPAPNEKGLKDTVKVPPGSVTRIIATFAPYSGRYVWHCHILEHEDYDMMRPLEVTDVRHQ

SEQ ID NO: 29 - protein, 509 aa, Bacillus spec.
MELEKFVDPMPIMKTAIPKKTSKDGDYYEIEMKEFSQKLHRDLNPTRLWGYDGQFPGPTIEVMRGKPARIKWMNNLPDTHFLPIDRSIHHVAHEPEVRTVVHLHGSETTPASDGYPEAWFTKDFAEVGSFFEQETYEYPNDQRAATLWYHDHAMGITRLNVYAGLSGLYIIRDPREEQLNLPKGEFDIPLLIQDRSFNDDGSLFYPAQPANPAPNLPNPSVLPFFVGDTILVNGKVWPYLQVEPRKYRFRILNGSNSRSYQLALDSEAPFYQIASDGGLLRRTVSLQAFDIRPAERIEAIIDFSKFEGQTITLKNNASTDATADVMQFQVVLPLSGEDTSIIPQNLSYIPSLQQNDVKRIRNLKISGTTDEYGRPLLLLNNKLWSDPVEEKPCLGTTEIWSFVNVTNVPHPMHIHLVQFQLLDHRAFNVELYNENGQIELVGPTIPPKINERGWKDTITAPAGQITRVIARFAPFSGYYVWHCHILEHEDYDMMRPFVVIDPKTEKERR

SEQ ID NO: 30 - protein, 619 aa, Bacillus spec.
MSLPINPSDPATIPKFVDALQKPVTAKPKYCRGQKKHDYYELVMMEGEHRFHKHFPNTLIWGYNGLYPGPTIEATKDKPIYVKYKNQLPLQHFLPVDFTLHAANDSQEVRTVTHLHGANVDWESDGHPEAWYTRDYRHTGPKFNKEIHEYTNHQPGTTMWYHDHAMALTRLNVYAGLAGFYLLRDALEERSNLPCGEYEYPILIQDKSFNEDGSLFYPDEPPFPVTVHPSITPGFVGNTIVVNGKLWPFLDVEPRKYRFRFLNGSNRRGYAISLSNEQTMMQIGTDGGFLSAPQAIQSVELLPAERTDIIIDFSACEGQEITLLNTDTDFIDEHTNVIMQFKVSLPLKCEDTSEIPERLAVTMDLHPHHAHIDRQLPLTATTDEFGRPMLLLNNRMYHDPATEKPSLDSIEVWSFINTTPFIHPIHLHLIQFKILERRPFDLELYQNEGIVSFTGPPEEPRDYERGWKDTVKVDAGKVTKIIMQWKDHVGNYMWHCHFLEHEDHDMMRPILVMKDVHAVQQPHAEIEQPTHHEATITSTTTAPTPTTNTTSTTHHNESPAPLLNASQIPFTEEEEIVDSLAEETTHIGNNDQHRPILLFPTPSTRTDTNRPRPRRPRRF

SEQ ID NO: 31 - protein, 510 aa, Bacillus spec.
MNLEKFVDELPIPEVAKPVKKNPKQTYYEIAMEEVFLKVHRDLPPTKLWTYNGSLPGPTIHANRNEKVKVKWMNKLPLKHFLPVDHTIHEGHHDEPEVKTVVHLHGGVTPASSDGYPEAWFSRDFEATGPFFEREVYEYPNHQQACTLWYHDHAMALTRLNVYAGLAGFYLISDAFEKSLELPKGEYDIPLMIMDRTFQEDGALFYPSRPNNTPEDSDIPDPSIVPFFCGETILVNGKVWPYLEVEPRKYRFRILNASNTRTYELHLDNDATILQIGSDGGFLPRPVHHQSFSIAPAERFDVIIDFSAYENKTITLKNKAGCGQEVNPETDANIMQFKVTRPLKGRAPKTLRPIFKPLPPLRPCRADKERTLTLTGTQDKYGRPILLLDNQFWNDPVTENPRLGSVEVWSIVNPTRGTHPIHLHLVQFRVIDRRPFDTEVYQSTGDIVYTGPNEAPPLHEQGYKDTIQAHAGEVIRIIARFVPYSGRYVWHCHILEHEDYDMMRPMDIIQ

SEQ ID NO: 32 - protein, 556 aa, Bacillus spec.
MSFKKFVDALPIPPVLKAEGNHDGVPFYEVTMKQVKQKLHRDLPPTTVWGYNGMYPGPTFEAERNHPILVKWKNKLPFEHLLPVDQTVHGAESSIPSVRTVVHLHGGRVRPESDGYPDAWFTRDFKNVGPKFVTEVYYYPNCQRPTTLWYHDHALGITRLNVYAGLAGFYLLRDEEEKKLNLPSGNFEIPLVIQDRSFYANGELFYPSQPGNMPPPAPQPPPPIDPTLPNPSVVPEFFGNTILVNGKVWPYLEVEPRKYRFRILNGSNSRFYRMKLSSGQNFVQIGTDGGLLEKPVLVSEIILAPAERADVIVDFSNHNGQSIILTNDAPTPFPDGESPSEDLRQIMQFRVKQTYCKHDKSKIPEKLSCLEQLNPQDAVITRKNTLLEAIDSFGRPMPLLNNMEWNQPITETPYNGTIEIWELYNTTPDTHPIHLHLVNFQILNRAVFTGDPNGSDLKVGPPIPPDPNERGWKDTVRANPGEVTRIIARFGPFTGTYPWHCHILEHEDYDMMRPYEVLANPSFNPCERDPEQCSDNAFSQCFGCYQQRKNKKEEKS

SEQ ID NO: 33 - protein, 527 aa, Bacillus spec.
MSFKKFVDALPIPPVLKAEGTHDGVPFYEVTMKQVKQKLHRDLPPTTVWGYNGMYPGPTFEAERNHPILVKWKNKLPFEHLLPVDQTVHGSESSIPSVRTVVHLHGGRVRPESDGYPDAWFTRDFKNVGPKFVTEVYYYPNCQRPTTLWYHDHALGITRLNVYAGLAGFYLLRDAEEKKLNLPSGNFEIPLVIQDRSFYANGELFYPSQPGNTPPPAPQPPPPIDPTLPNPSVVPEFFGNTILVNGKVWPYLEVEPRKYRFRILNGSNSRFYRMKLSSGQNFVQIGTDGGLLEKPVLVSEIILAPAERADVIVDFSNHNGQRIILTNDAPTPFSDGESPSEDLRQIMQFRVKQTCCKPDKSKIPEKLSCLELLNPQDAVITRKNTLLEATDNFGRPMPLLNNMEWNQPITETPYNGTIEIWELYNTTPDTHPIHLHLVNFQILNRAVFTGDPNGSNLKVGPPIPPDPNEKGWKDTVRANPREVTQIIARFGPFTGTYPWHCHILEHEDYDMMRPYQVLANPSFNPCE

SEQ ID NO: 34 - protein, 527 aa, Bacillus spec.
MSFKKFVDALPIPPVLKAEGTHDGVPFYEVTMKQVKQKLHRDLPPTTVWGYNGMYPGPTFEAERNHPILVKWKNKLPFEHLLSVDQTVHGAESSIPSVRTVVHLHGGRVRPESDGYPDAWFTRDFKNVGPKFVTEVYYYPNCQRPTTLWYHDHALGITRLNVYAGLAGFYLLRDEEEKKLNLPSGNFEIPLVIQDRSFYANGELFYPSQPGNMPPPAPQPPPPIDPTLPNPSVVPEFFGNTILVNGKVWPYLEIEPRKYRFRILNGSNSRFYRMKLSSGQNFVQIGTDGGLLEKPVLVSEIILAPAERADVIVDFSNHNGQSIILTNDAPTPFPDGESPSEDLRQITQFRVKQTYCKPDKSKIPEKLSCLEQLNPQDAVITRKNTLLEATDNFGRPMPLLNNMEWNQPITETPYNGTIEIWELYNTTPDTHPIHLHLVNFQILNRAVFTGDPNGSDLKVGPPIPPDPNERGWKDTVRANPGEVTRIIARFGPFTGTYPWHCHILEHEDYDMMRPYEVLANPSFNPCE

SEQ ID NO: 35 - protein, 497 aa, Bacillus spec.
MELKKFIDPLPIPSTIKPKGTYKGKPFYDVRMIETLHKFHSDLPKTKVWGYNGMVPGPTFEVKKDHPVYVRWSNDLPKKHFLPVDTTVHGAHDNPEVRTVVHLHGSPSEPDSDGHPEAWFTKNFQQTGPSFVKEIYHYSNRERATALWYHDHALGITRLNVYAGLAGLYLIRDKEEKSLPLPKGKYEIPLIVQDKSFNPDGSLFYPAQPEDPSPGLPYPSIVSLFLGNTIAVNGKVWPFLKVEPRKYRFRLLNASNTRTYKFQLSDLSSFFLIGTDGGLLKRPIKVQSLDVSPAERIDIIIDFSRFEGKKVVLQNGIDLDNPTGEIMEFQVTKRLSCPDKSKIPCKLSNIDFIPVEKVKKVRRLTLNDSEDEFGRLMLLLDNKEWADPVTETPLLNSVEIWELVNLTVGIHPIHIHLVNFQVLDRHDLNGNLLDSLPADFGLKDTVLVAGEQIVRIIMKFEPYSGDYVWHCHRLEHEDHDMMRPLKIIPSSLIKYKK

SEQ ID NO: 36 - protein, 497 aa, Bacillus spec.
MELKKFIDPLPIPSAIKPIGTYKGKPFYDVRMVETLHKFHSDLPKTKVWGYNGMVPGPTFEVKKDHPVYVRWSNDLPEKHFLPVDTTVHGAHDNPEVRTVVHLHGSPSEPDSDGHPEAWFTKNFQQTGPSFVKEIYHYSNRERATALWYHDHALGITRLNVYAGLAGLYLIRDKEEKSLPLPKGKYEIPLIVQDKSFNPDGSLFYPGKPEDPSPGLPYPSIVPLFLGNTIAVNGKVWPFLKVEPRKYRFRLLNASNTRTYKFQLSDLSSFFLIGTDGGLLKRPIKVQSLDVSPAERIDIIIDFSRFEGKKVVLQNGIDLDNPTGEIMEFQVTKRLPCPDKSKIPCKLSNIDFIPVEKVKKVRRLTLNDSEDEFGRLMLLLDNKEWDDPVTETPLLNSVEIWELVNLTVGIHPIHIHLVNFQVLDRHDLNGNLLDPLPADFGLKDTVLVAGEQTVRIIMKFEPYSGDYVWHCHRLEHEDHDMMRPLKIIPSSLNKHKR

SEQ ID NO: 37 - protein, 491 aa, Bacillus spec.
MELEKFVDSLPIPSIIKPKGTCQGKPLYDVRMIETLHKFHRDLPKTKVWGYNGLVPGPTFDVQKNHPVYVRWTNDLPKKHFLPVDKTVHGAHDTPEVRTVVHLHGNPSEPDSDGHPEAWFTRNFQQTGPRFVKEIYHYTNLERATALWYHDHALGIIRLNVYAGLAGLYLIRDKEERSLPLPKDKYEIPLIVQDKSFNPDGSLFYPAQPDNPSPNLPFPSIVPLFLGNTITVNGKVWPFLKVEPRKYRFRLLNASNTRTYKFQFSDLSSFFLIGTDGGLLKRPIQVQSLDVSPSERVDIIVDFSSLEGKKVVLQNESDLENPTGEVMEFQVTKPLSCPDKSKIPCLLSNIDFFPVKQVNVRKVRRLTLSDSEDEFGRPMLLLDNKEWDDPVTETPLLDSIEIWELVNLTAGIHPIHIHLVNFRVLDRHDLNGKLLPPLPADFGLKDTVLVEGEQIVRIIMKFEPYSGDYVWHCHRLEHEDHDMMRPLKIIK

SEQ ID NO: 38 - protein, 498 aa, Bacillus spec.
MELKKFIDPLPIPSAIKPIGTYKGKPFYDVRMIETLHKFHSDLPKTKVWGYNGMVPGPTFEVKKDHPVYVRWSNDLPEKHFLPVDTTVHGAHDNPEVRTVVHLHGSPSEPDSDGHPEAWFTKNFQQTGPSFVKEIYHYTNRERATTLWYHDHALGITRLNVYAGLAGLYLIRDKEEKSLPLPKGKYEIPLIVQDKSFNPDGSLFYPAQPEDPPPGLPYPSIVPLFLGNTITVNGKVWPFLKVEPRKYRFRLLNASNTRTYKFQLSDLSSFFLIGTDGGLLKRPIKVQSLDVSPAERIDIIIDFSRFEGKKVVLQNGIDLDNPTGEIMEFQVTKPLSCPDKSKIPCKLSNIDFIPVEKVKKVRRLTLNDSEDEFGRLMLLLDNKEWDDPVTETPLLNSVEIWELVNLTVGIHPIHIHLVNFQVLDRHDLNGNLLNPLPADFGLKDTVLVAGEQTVRIIMKFEPYSGDYVWHCHRLEHEDHDMMRPLKIIPSNLNRHKLN

SEQ ID NO: 39 - protein, 522 aa, Bacillus spec.
MSPNLEKFVDRLPLAEKIRPVREEGGIAYYEVTMEEFWQKLHRDLRPTRLWGYNRSFPGPLFDVPHGKKIRVKWTNHLPQRHFLPMDTTILDEMGTDFPEVRTVVHLHGGETEPDSDGYPEAWFTRDFNKTGPDFKKEVYEYTNSQRPATLWYHDHAIGITRLNVYAGLAGMYIIRDPKEKAFHLPSGKYEIPLLLTDRTFNNDGSLFYPRQPQNPGPETPDPSVVPFFLGDTILVNGKVWPYLEVEPRKYRFRIVNASNTRAYRLYLDSGQAFYQIGTDGGLLRRPVQVENLALEPAERADLILDFSEYAGQTILLKNDLGPNADPADQTGDVMQFRVVLPVAGEDTSRIPRSLSSIPVPSSHNVSAIRHLKLTGATDSYGRPLLLLDKKRWMDPVTETPRLGTTEIWSLANTTAFTHPIHIHLIQFQILDRRPFDLDLYNETGQIVYTGPATPPEPNERGFKDTVAAPGGQITRVMMRFSPYAGDYVWHCHILEHEDYDMMRPFQVIDPDLPASDGPLLD

SEQ ID NO: 40 - protein, 553 aa, Bacillus spec.
MVSSEPNEKASKIELEENAEDKVEVKCCNDNICFIVNPSDPFTIPKFIDKLLIPETAKPIDDPNNEYKDNEKHYHITMKEAEHYFSSYFPPTKIWGYNGLYPGPTIEAFKNISTHVLWDNKLPDKHFLPYDTTLHGTVDDPEGKTVVHLHGAKVESSSDGHPEAWFTNGFKVTGPEFTRKVYEYTNYQSGTTLWYHDHAMGQTRLNVYAGLAGFYILRDLLEERLNLPKGKYEVPLLVQDKSFTEEGELFYPAVPPFPVSVFPSIVPGFFGDTIVVNGKVWPYLDVEPRKYRFRILNASNRRQYIMRLSNNEEFIQIGTDGGLIEEPVNLTSFKLMPAERIDIIIDFSQHVGERIILMNDDTNADISGTNMIMRFNVVKPLQGEDTSVVPSTLRVVHPLNKGLVRRKRKVSFGAMTDRYGRPMLMIDNMTWSDPVTEKPELDSIEIWNLINPMPVPHPIHIHLIQFKILWRRPFDVEAYNNTGEVIYTGPKEEPHDYERGFKDTVDSEAGKVTRIIMQFKGYAGNYVWHCHFLEHEDYEMMRPLKVVECNCVK

SEQ ID NO: 41 - protein, 513 aa, Bacillus spec.
MTLEKFADALPIPDTLKPVQQTTEKTYYEVTMEECAHQLHRDLPPTRLWGYNGLFPGPTIEVKRNENVYVKWMNNLPSEHFLPIDHTIHHSDSQHEEPEVKTVVHLHGGVTPDDSDGYPEAWFSKDFEQTGPYFKREVYHYPNQQRGAILWYHDHAMALTRLNVYAGLVGAYIIHDPKEKRLKLPSGEYDVPLLITDRTINEDGSLFYPSGPENPSPSLPKPSIVPAFCGDTILVNGKVWPYLEVEPRKYRFRVINASNTRTYNLSLDNGGEFIQIGSDGGLLPRSVKLNSFSLAPAERYDIIIDFTAYEGQSIILANSEGCGGDANPETDANIMQFRVTKPLAQKDESRKPKYLASYPSVQNERIQNIRTLKLAGTQDEYGRPVLLLNNKRWHDPVTEAPKAGTTEIWSIVNPTQGTHPIHLHLVSFRVLDRRPFDIARYQERGELSYTGPAVPPPPSEKGWKDTIQAHAGEVLRIAVTFGPYSGRYVWHCHILEHEDYDMMRPMDITDPRK

SEQ ID NO: 42 - protein, 618 aa, Bacillus spec.
MQINPSDPATIPKFVDELPKPMIAKPKYSRGQQKNDYYELVMMEGQHRFHKHFPNSLIWGYNGLYPGPTIEASKDKTIYVKYKNQLPLQHFLPVDFTLHAANDSQEVRTVTHLHGANVDWQSDGHPEAWYTKDYRHTGPKFNKEIHEYTNHQPGTTMWYHDHAMALTRLNVYAGLAGFYLLRDALEERSNLPCGDYEYPILIQDKSFNEDGSLFYPDEPPFPVPVHPSITPGFVGNTIVVNGKLWPFLDVEPRKYRFRLLNGSNRRGYVISLSNDQPMFQIGTDGGFLSAPQLIQSVELLPAERTDVIIDFSSCAGQEITLLNTDTDFSDEHTNVIMQFRVILPLKCEDTSEIPERLAVSMDLHPHHAHIERQLPLTATTDEFGRPMLLLNDRMYHDPATEKPSLDSVEIWNFINATPFIHPIHLHLIQFKILERRPFDLDIYQNEGIVQFTGPPEEPRDYERGWKDTVKADAGKVTKIIMHWKDHVGNYMWHCHFLEHEDHDMMRPILVMKDVHAVQQPHAAVEHPVTHQESTAPTNPTSTTNPTSTTHHSESSAPLLNEVEHSVLEEEKEALPNEEESITIHKGDNTRHMPTLLLPTLPTNTPTNRQRPRRRTRRF

SEQ ID NO: 43 - protein, 513 aa, Bacillus spec.
MKLEKFVDRLPIPQVLQPQSKSKEMTYYEVTMKEFQQQLHRDLPPTRLFGYNGVYPGPTFEVQKHEKVAVKWLNKLPDRHFLPVDHTLHDDGHHEHEVKTVVHLHGGCTPADSDGYPEAWYTKDFHAKGPFFEREVYEYPNEQDATALWYHDHAMAITRLNVYAGLVGLYFIRDREERSLNLPKGEYEIPLLIQDKSFHEDGSLFYPRQPDNPSPDLPDPSIVPAFCGDTILVNGKVWPFAELEPRKYRFRILNASNTRIFELYFDHDITCHQIGTDGGLLQHPVKVNELVIAPAERCDIIVDFSRAEGKTVTLKNRIGCGGQDADPDTDADIMQFRISKPLKQKDTSSLPRILRKRPFYRRHKINALRNLSLGAAVDQYGRPVLLLNNTKWHEPVTETPALGSTEIWSIINAGRAIHPIHLHLVQFMILDHRPFDIERYQENGELVFTGPAVPPAPNEKGLKDTVKVPPGSVTRIIATFAPYSGRYVWHCHILEHEDYDMMRPLEVTDVRHQ

SEQ ID NO: 44 - protein, 558 aa, Bacillus spec.
MSLKKFVDALPIPPVIEPKGKRDGIPFYEVKMRQIQQKLHRDLPPTTVWGYNDMYPGPTFNVRRNRPILVKWENKLPYQHLLPVDTTVHGADPSQPSVRTVTHLHGGRVRPENDGYPEAWFTKNYENVGSKFLHEVYYYPNCQRPTTLWYHDHALGITRLNVYAGLAGFYLIRDQKEEKLNLPRGKYEIPLVIQDRSFYPDGQLFYPSQPGQEPPPAPQPPPPTDPDLPNPSVVPEFFGNTILVNGKVWPYLEVQPRKYRFRILNGSNSRFYRMRLSSGQDFIQIGTDGGLLKKPVPISEIILAPAERADVIIDFSRHKGENIILTNDAPAPFPDGTPPSPDISQIMEFRVKQKGFKKDNSKVPKRLSCLEKLDPKDAITVRKNFLVESTDQFGRPLLLLNNMRWFDLITETPYNGTIEIWELYNVTPDTHPIHLHLIQFQILNRAPITVDENGNIIDVGKAKEPDPNEMGWKDTVRANPGEVTRIIARFGPFTGTYVWHCHILEHEDHDMMRPYEVLKNHDFDPCIPIRGVCRDDSFTQCCHEDDENCKCKERNPKK

SEQ ID NO: 45 - protein, 609 aa, Bacillus spec.
MEGCDMMDLTPFVDSLPIPQYISPKERHTCFTYYEIAMKEFYHQFHSELAPTKIWGYGGQFPGPVINVHRGECTHVKWMNQLPDKHLLPIDRTLHGSGEHMPDVRTVVHLHGAEVEPESDGYPEAWFTNDFKFVGPVFDSPIYKYNNNQRASTLWYHDHAVGITRLNVYAGLVGMYIIRDEEERKLHLPSGAYEIPLVIADRGFNEDGSLFYPDTTNVRPGDLEPGLEFPHPSVTPGETFENITVNGKVWPFLEVERRKYRFRILNASNERFYRIKLSNGQKFIQIGTDGGLLEKSVHMTELTIGPSERMDVVLDFSKYQPGTEIVIENTARSPFDFDPPVGIDPDPKKDGQIMQFRVVELDGTDTSKVPAVLSRIPKLKACDAERTRDITLEAEIDQYGRFRFLLNKKGFMERIDVKPKLNDTEIWRFINTAGATHPMHIHLIQFQILDRIPFDVQGFTANGLLSFTGPPQPPAENEHGWKDTVQSPPGFVTRVIMRFAPFTGRYVYHCHILEHEDYDMMRPFEIVERRCVCNQNCSHKCTCKCECKKTCTCTGQCKCICKQPDKCECKTTCKCHEKKPSQPPKDVECQSQKCPKCPPCSETYRCECE

SEQ ID NO: 46 - protein, 539 aa, Bacillus spec.; identical to SEQ ID NO: 2

SEQ ID NO: 47 - protein, 513 aa, Bacillus spec.; identical to SEQ ID NO: 3

SEQ ID NO: 48 - protein, 513 aa, Bacillus spec.; identical to SEQ ID NO: 1

SEQ ID NO: 49 - protein, 21 aa, Bacillus subtilis
IVNPTQGTHPIHLHLVSFRVL

SEQ ID NO: 50 - protein, 21 aa, Bacillus subtilis
IINPTRGTHPIHLHLVSFRVL

SEQ ID NO: 51 - protein, 21 aa, Escherichia coli
SGVGDMMLHPFHIHGTQFRIL

SEQ ID NO: 52 - protein, 21 aa, Streptomyces coelicolor
MITHGEYYHTFHMHGHRWADN

SEQ ID NO: 53 - protein, 20 aa, Trametes trogii
AAAPGFPHPFHLHGHTFAVV

SEQ ID NO: 54 - protein, 14 aa, artificial sequence
  Consnesus sequence
IIHPFHLHGSFRVL

SEQ ID NO: 55 - protein, 23 aa, Bacillus subtilis
GPWFWHCHILEHEDYDMMRPMDI

SEQ ID NO: 56 - protein, 23 aa, Bacillus subtilis
GRYVWHCHILEHEDYDMMRPMDI

SEQ ID NO: 57 - protein, 23 aa, Escherichia coli
HAYMAHCHLLEHEDTGMMLGFTV

SEQ ID NO: 58 - protein, 23 aa, Streptomyces coelicolor
GAWMYHCHVQSHSDMGMVGLFLV

SEQ ID NO: 59 - protein, 23 aa, Trametes trogii
GPWFLHCHIDFHLEAGFAVVMAE

SEQ ID NO: 60 - protein, 18 aa, artificial sequence
  Consnesus sequence
GWMWHCHILEHEDGMMMI

SEQ ID NO: 61 - protein, 11 aa, Bacillus subtilis
YGRPVQLLNNK

SEQ ID NO: 62 - protein, 11 aa, Bacillus subtilis
YGRPVLLLNNK

SEQ ID NO: 63 - protein, 11 aa, Escherichia coli
VRKLQLSMDPM

SEQ ID NO: 64 - protein, 11 aa, Streptomyces coelicolor
THTIVFNDMTI

SEQ ID NO: 65 - protein, 11 aa, Trametes trogii
FNGTNFFINGA

SEQ ID NO: 66 - protein, 5 aa, artificial sequence
  Consnesus sequence
YRVLN

(19) United States
(12) Patent Application Publication
(10) Pub. No.: US 2015/0159144 A1
(43) Pub. Date: Jun. 11, 2015

(54) ENZYME VARIANTS WITH IMPROVED PROPERTIES

(71) Applicants: Klara Birikh, Kaarina (FI); Alexey Azhayev, Kaarina (FI)

(72) Inventors: Klara Birikh, Kaarina (FI); Alexey Azhayev, Kaarina (FI)

(73) Assignee: METGEN OY, Kaarina (FI)

(21) Appl. No.: 14/344,028

(22) PCT Filed: Sep. 13, 2012
(86) PCT No.: PCT/FI2012/050884

§ 371 Date: Nov. 24, 2014

Related U.S. Application Data
(60) Provisional application No. 61/535,032, filed on Sep. 15, 2011.

(51) Int. Cl. C12N 9/02; D21C 5/00; C02F 3/34; D06M 16/00

(52) U.S. Cl. CPC C12N 9/0061; D06M 16/003; D21C 5/005; C02F 3/342; C02F 2305/023

(57) ABSTRACT

The present invention relates to laccase variants having improved enzymatic properties in alkaline conditions and uses thereof as eco-friendly biocatalysts in various industrial processes.

### FIELD OF THE INVENTION

The present invention relates to laccase variants and uses thereof as eco-friendly biocatalysts in various industrial processes.

### BACKGROUND OF THE INVENTION

Laccases (EC 1.10.3.2) are enzymes having a wide taxonomic distribution and belonging to the group of multicopper oxidases. Laccases are eco-friendly catalysts, which use molecular oxygen from air to oxidize various phenolic and non-phenolic lignin-related compounds as well as highly recalcitrant environmental pollutants, and produce water as the only side-product. These natural “green” catalysts are used for diverse industrial applications including the detoxification of industrial effluents, mostly from the paper and pulp, textile and petrochemical industries, use as bioremediation agent to clean up herbicides, pesticides and certain explosives in soil. Laccases are also used as cleaning agents for certain water purification systems. In addition, their capacity to remove xenobiotic substances and produce polymeric products makes them a useful tool for bioremediation purposes. Another large proposed application area of laccases is biomass pretreatment in biofuel and pulp and paper industry.

Laccases have a wide substrate specificity and they can oxidize many different substrate compounds. Owing to chemical properties of the substrates, they become more readily oxidized in different pH conditions, either alkaline or acidic. On the other hand, the advantageous pH range of action of different laccases may vary, which means that they have a preference to substrates within that range. For instance, relatives of CotA laccase are known to work best in acidic conditions.

A wider operable pH range would be an important feature in laccases, especially in waste water and remediation applications, as acidity of these environments may vary significantly. This feature is also critical for biomass pre-treatment processes, which in certain cases are carried out under alkaline conditions. Thus, there is an identified need in the art for developing laccase variants having a wider pH range of action.

### BRIEF DESCRIPTION OF THE INVENTION

In one aspect, the present invention relates to laccase variants, which comprise a glutamine residue situated within 6 Angstrom (A) distance to the type 1 Copper ion in the 3-dimentional structure of the laccase variant.

In some embodiments, the laccase variant may comprise an amino acid sequence showing at least 50% identity to an amino acid sequence selected from the group consisting of SEQ ID NO:3, SEQ ID NO:4, and SEQ ID NO:5, comprising at least one amino acid variant selected from the group consisting of glutamine (Q) in a position which corresponds to the position 386 of the amino acid sequence depicted in SEQ ID NO:3 and a Proline-Tryptophan-Phenylalanine (PWF) sequence in a position which corresponds to the position 487-489 of the amino acid sequence depicted in SEQ ID NO:3.

In other embodiments, the present laccase laccase variants have an increased enzymatic activity in alkaline conditions as compared to that of a corresponding control enzyme lacking said amino acid variants.

The present invention also relates to nucleic acid molecules encoding the present laccase variants, vectors comprising said nucleic acid molecules, and recombinant host cells comprising said vector.

In other aspects, the invention relates to a method of producing the present laccase variants. The method comprises the steps of i) culturing a recombinant host cell according to the present invention under conditions suitable for the production of the laccase variant, and ii) recovering the laccase variant obtained.

In further aspects, the invention relates to various uses of the present laccase variants, especially in pulp delignification, textile dye bleaching, wastewater detoxifixation, and xenobiotic detoxification.

Other specific embodiments, objects, details, and advantages of the invention are set forth in the dependent claims, following drawings, detailed description and examples.

### BRIEF DESCRIPTION OF THE DRAWINGS

In the following the invention will be described in greater detail by means of preferred embodiments with reference to the attached drawings, in which

FIG. 1 is a schematic representation of T1 (Cu1) and T2/T3 (Cu4/Cu2-Cu3) copper sites of laccase CotA from Bacillus subtilis with indicated distances between the most important atoms (adopted from Enguita et al., “Crystal Structure of a Bacterial Endospore Coat Component”, J. Biol. Chem., 278, 19416-19425, 2003). The area of MUT1 mutation is indicated by the dashed oval.

FIG. 2 shows the three-dimentional structure of Cu1 site ligand environment in radius of 6A elucidated from crystal structures of four evolutionary distant laccase (Bacillus Subtilis COTA protein, Streptomyces Coelicolor laccase, E. coli CuEO laccase, and Trametes Trogii laccase). Respective accession numbers in Structure Data Base 1UVW, 3KW8, 2FQD and 1KYA. Numeration of residues in B. subtilis laccase crystal structure is 9 residues less than that of the full size protein (a small N-terminal fragment was missing from the crystallized protein). A residue corresponding to the Glutamine 368 is depicted in black.

FIG. 3 shows an alignment of the two conserved regions containing T1 copper ligands derived from evolutionary distant laccases (Bacillus Subtilis COTA protein, Streptomyces Coelicolor laccase, E. coli CuEO laccase, and Trametes Trogii laccase). Corresponding crystal structures of the Cu1 surrounding are presented in FIG. 2. Empty arrows indicate the positions of Cu-1 ligands, black arrow indicates axial ligand. Panel C shows just a list of the sequences surrounding Q386 substitution (M1) identified from the 3-D structures. M1 position is framed.

FIG. 4 shows a multiple alignment of amino acid sequences that are related to COT1 (SEQ ID NO:1) and COT2 (SEQ ID NO:2) and were identified in a Blast search.

FIG. 5 shows a schematic representation of introducing MUT1 into Laccase type2 gene from Bacillus pseudomycoides. Primers 1 and 2 represent terminal regions of the recombinant gene. Primers 3 and 4 represent fragments of the top and bottom strands of the mutated gene surrounding mutation site (X on the primers depicts the mutation). PCR reactions (1) and (2) produce two overlapping fragments of the gene (Fragment 1 and Fragment2), both bearing the mutation. The third PCR reassembles the full length gene with mutation (black bar) at the desired position.

FIG. 6 illustrates measurements of relative activity of the present laccases at different pH. Panel A demonstrates the selection of the initial rate time range for the reactions. As this time depends on the amount of the enzyme in the reaction, a suitable dilution of the enzyme needs to be obtained for convenient measurement. In the present examples, 10 min time was within the linear range in all pH conditions. Panel B illustrates the photometric measurement of ABTS absorbance; maximal initial rates of the present laccases (with and without mutation—WT and Mutant, respectively).

### DETAILED DESCRIPTION OF THE INVENTION

The present invention is based on a surprising finding that certain amino acid substitutions result in increased laccase activity especially in alkaline conditions.

The term “amino acid substitution” is used herein the same way as it is commonly used, i.e. the term refers to a replacement of one or more amino acids in a protein with another. Artificial amino acid substitutions may also be referred to as mutations.

As used herein, the term “alkaline” is a synonym for the term “basic”. Thus, the term “alkaline conditions” refers to conditions having a pH value greater than 7.

The term “laccase activity” is used herein to mean maximal initial rate of the oxidation reaction. Laccase activity may be determined by standard oxidation assays known in the art including, but not limited to measurement of oxidation of Syringaldazine by laccase according to Sigma online protocol, or according to Cantarella et al. (“Determination of laccase activity in mixed solvents: Comparison between two chromogens in a spectrophotometric assay”, Biotechnology and Bioengineering V. 82 (4), pp 395-398, 2003). An example of determining relative laccase activity at different pH is presented in Example 2. Any substrate suitable for the enzyme in question may be used in the activity measurements. A non-limiting example of a substrate suitable for use in assessing the enzymatic activity of the present laccase variants is 2,6-Dimethoxyphenol (2,6-DMP).

As used herein, the term “increased (or improved) laccase activity” refers to a laccase activity higher than that of a corresponding non-mutated laccase enzyme under the same conditions. That is to say, for instance if enzymes A and B have equal activity at pH5, whereas at pH 9 the same preparation of enzyme A has a higher activity than that of enzyme B, then enzyme A is denoted as a laccase variant having “an increased laccase activity in alkaline conditions”. Certain amino acid variants in certain positions of laccase protein disclosed herein result in increased laccase activity at alkaline pH at least by 50% as compared to the corresponding laccase enzymes omitting this amino acid variant. In some embodiments, an increased laccase activity in alkaline conditions means about 2-fold, and preferably 5-fold, higher laccase activity as compared to that of a corresponding non-mutated variant.

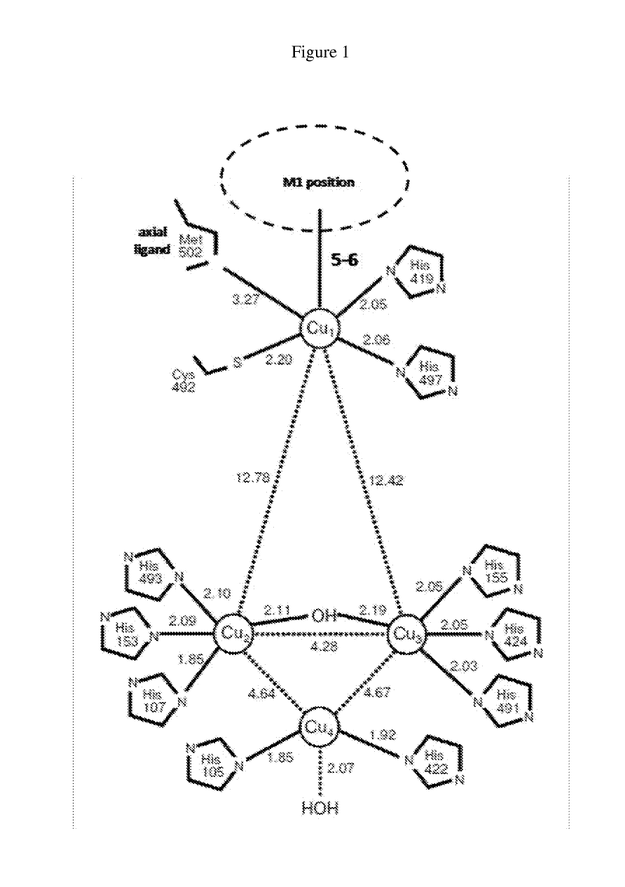

Laccase molecules are usually monomers consisting of three consecutively connected cupredoxin-like domains twisted in a tight globule. The active site of laccases contains four copper ions: a mononuclear “blue” copper ion (T1 site) and a three-nuclear copper cluster (T2/T3 site) consisting of one T2 copper ion and two T3 copper ions (FIG. 1).

Laccases isolated from different sources are very diverse in primary sequences; however, they have some conserved regions in the sequences and certain common features in their three-dimensional structures. A comparison of sequences of more than 100 laccases has revealed four short conservative regions (no longer than 10 aa each) which are specific for all laccases (Kumar et al., “Combined sequence and structure analysis of the fungal laccase family”, Biotechnol. Bioeng., 83, 386-394, 2003; Morozova et al., “Blue laccases”, Biochemistry (Moscow), 72, 1136-1150, 2007). One cysteine and ten histidine residues form a ligand environment of copper ions of the laccase active site present in these four conservative amino acid sequences.

The T1 site of the enzyme is the primary acceptor of electrons from reducing substrates. The potential of the enzyme T1 site also determines the efficiency of catalysis on oxidation of the majority of laccase substrates, and therefore T1 site is primary target for laccase protein engineering. The T1 site has as ligands two histidine imidazoles and the sulfhydryl group of cysteine, which form a trigonal structure (FIG. 1). The fourth residue in the immediate proximity of the copper 1 is so called axial ligand—methionine or phenylalanine (Met502 in FIG. 1). These ligands in the primary sequence are situated in the two conserved regions (third and fourth) at the distal end of the protein.

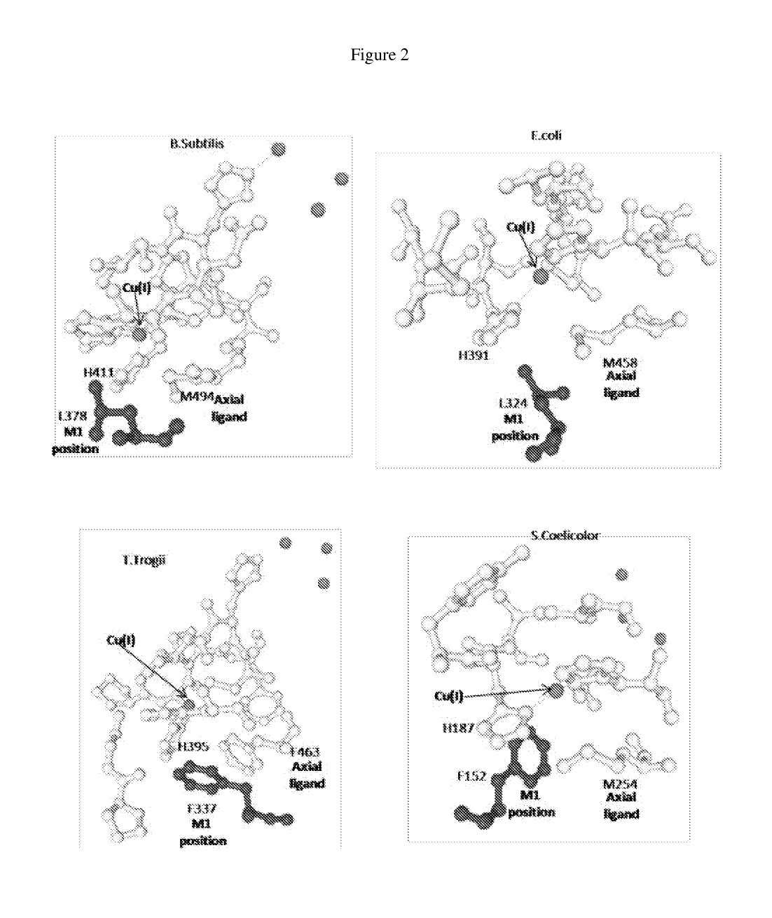

Most residues forming the ligand environment of the type 1 copper ion are relatively conserved and three-dimensional structures of the copper binding domains from remotely related laccases may be very similar. As an example, FIG. 2 shows surrounding of copper 1 atom in 6 A radiuses of four evolutionary very distant laccases (sequence identity not more than 20%, length of the protein chain varies from 273 to 503 aa). All residues comprising copper 1 environment in these laccases are adjacent or proximal in the primary sequence to the copper ligands (two histidines and the cystein) and belong to the conserved regions, with one exception. A residue (marked dark in the FIG. 2, usually hydrophobic, in the depicted cases leucine or phenylalanine) is protruding into the environment of copper 1 atom from a distant part of the primary sequence.

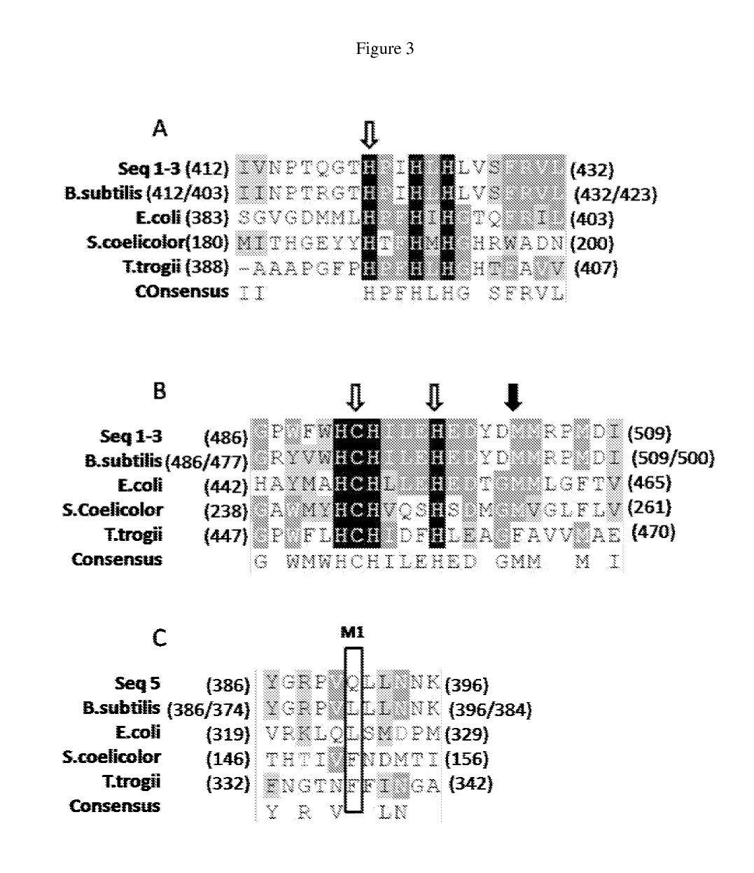

It has now been surprisingly found that when the protruding amino acid is substituted by Glutamine (Q), the result is an improved laccase performance at alkaline conditions. This substitution is hereinafter referred to as MUT1, or M1. This position is situated in the part of the primary sequence which is not conserved between distant laccases. FIG. 3 shows fragments of aligned primary sequences of the laccases from FIG. 2. All residues depicted in the crystal structures in fair grey are situated in the regions depicted in panels A and B (conserved regions). Whereas the residue depicted in crystal structures black (FIG. 2, M1 position) is situated in the regions depicted on panel C. Panel C was not generated by alignment protocols owing to lack of sufficient homology in these part of the sequences), but the panel is only a list of sequences surrounding the MUT1 position elucidated from 3-D structure (marked black in FIG. 2).

In other examples where laccases in question are more homologous, this region may be sequentially conserved, and thus MUT1 position may be elucidated from a sequence alignment. Whether sequentially conserved or not, this residue can be unambiguously identified in practically any laccase by being present in an about 5-6 A radius of copper 1 in proximity to the axial ligand of Copper 1 atom. To our best knowledge there is no glutamine in the copper-1 5-6 A environment in any of the laccases with a known three-dimensional structure.

In connection with the present invention, two laccase protein sequences COT1 (SEQ ID NO:1) and COT2 (SEQ ID NO:2) absent from publicly available databases were cloned from laboratory strains of Bacillus subtilis. In-silica analysis of the protein structures together with intensive experimental research using combinatorial methods of molecular biology confirmed that an artificial Leucine (L) to Glutamine (Q) substitution in position 386 of both SEQ ID NO:1 and SEQ ID NO:2 improved laccase performance at alkaline conditions. Improved performance was also achieved by another mutation, i.e. adjacent Arginine (R) 487 to Proline (P), Tyrosine (Y) 488 to Tryptophan (W) and Valine (V) 489 to Phenylalanine (F) triple substitution, either alone or in combination with the L386Q substitution. Amino acid sequence of a laccase variant comprising both of these mutations is depicted in SEQ ID NO:3, whereas amino acid sequences depicted in SEQ ID NO: 4 and SEQ ID NO:5 represent laccase variants comprising only the L386Q substitution or the triple substitution, respectively.

Amino acid variants presented by these mutations appear to be unique at corresponding positions among related polypeptide sequences since they were not identified in a protein search in BLAST, a public internet service which compares the query sequence to all sequences deposited in the public domain. The search revealed some closely related sequences only a few amino acids different from the queries and a whole range of homologous sequences with different degree of similarity (Table 1).

TABLE 1
The results of the Blast search
The sequences (accession numbers) are listed in the order of
decreasing similarity.

| | | | | M1-3ple |
|---|---|---|---|---|
| Accession | Description | Identity % | Similarity % | Q . . . PWF |
| YP_004206641.4 | spore copper-dependent laccase [Bacillus | 98 | 99 | L . . . RYV |
| | subtilis BSn5] bj\|BAI84141.1\|spore coat protein | | | |
| | A [Bacillus subtilis subsp. natto BEST195] | | | |
| | >gb\|ADV95614.1\|spore copper-dependent | | | |
| | laccase [Bacillus subtilis BSn5]spore copper- | | | |
| | dependent laccase [Bacillus subtilis subsp. | | | |
| | subtilis str. 168] >ref\|ZP_03590314.1\|spore coat | | | |
| | protein (outer) [Bacillus subtilis subsp. subtilis | | | |
| | str. 168] >ref\|ZP_03594593.1\|spore coat protein | | | |
| | (outer) [Bacillus subtilis subsp. subtilis str. NCIB | | | |
| | 3610] >ref\|ZP_03599005.1\|spore coat protein | | | |
| | (outer) [Bacillus subtilis subsp. subtilis str. | | | |
| | JH642] >ref\|ZP_03603283.1\|spore coat protein | | | |
| | (outer) [Bacillus subtilis subsp. subtilis str. SMY] | | | |
| | >sp\|P07788.4\|COTA_BACSU RecName: | | | |
| | Full = Spore coat protein A >pdb\|1GSK\|A Chain | | | |
| | A, Crystal Structure Of Cota, An | | | |
| NP_388511.1 | Endospore Coat Protein From Bacillus Subtilis | 98 | 99 | L . . . RYV |
| | >pdb\|1OF0\|A Chain A, Crystal Structure Of | | | |
| | Bacillus subtilis Cota After 1 h Soaking With Ebs | | | |
| | >pdb\|1UVW\|A Chain A, Bacillus subtilis Cota | | | |
| | Laccase Adduct With Abts >pdb\|1W6L\|A Chain | | | |
| | A, 3d Structure Of Cota Incubated With Cucl2 | | | |
| | >pdb\|1W6W\|A Chain A, 3d Structure Of Cota | | | |
| | Incubated With Sodium Azide >pdb\|1W8E\|A | | | |
| | Chain A, 3d Structure Of Cota Incubated With | | | |
| | Hydrogen Peroxide >pdb\|2BHF\|A Chain A, 3d | | | |
| | Structure Of The Reduced Form Of Cota | | | |
| | >pdb\|2X88\|A Chain A, Crystal Structure Of | | | |
| | Holocota >emb\|CAB12449.1\|spore copper- | | | |
| | dependent laccase [Bacillus subtilis subsp. | | | |
| | subtilis str. 168] | | | |
| BAA22774.1 | spore coat proein A [Bacillus subtilis] | 98 | 99 | L . . . RYV |
| ACS44284.1 | spore coat protein [Bacillus subtilis] | 98 | 98 | L . . . RYV |
| 2X87_A | Chain A, Crystal Structure Of The Reconstituted | 98 | 99 | L . . . KTV |
| | Cota | | | |
| 2WSD_A | Chain A, Proximal Mutations At The Type 1 Cu | 97 | 98 | L . . . RYV |
| | Site Of Cota-Laccase: I494a Mutant | | | |
| ACM46021.1 | laccase [Bacillus sp. HR03] spore copper- | 97 | 98 | L . . . RYV |
| | dependent laccase [Bacillus subtilis subsp. | | | |
| | spizizenii ATCC 6633] >ref\|YP_003865004.1\| | | | |
| | spore copper-dependent laccase (outer coat) | | | |
| | [Bacillus subtilis subsp. | | | |
| ZP_06872569.1 | spizizenii str. W23] >gb\|EFG93543.1\|spore | 96 | 97 | L . . . RYV |
| | copper-dependent 1031 | | | |
| | laccase [Bacillus subtilis subsp. spizizenii ATCC | | | |
| | 6633] >gb\|ADM36695.1\|spore copper- | | | |
| | dependent laccase (outer coat) [Bacillus subtilis | | | |
| | subsp. spizizenii str. W23] | | | |
| AAB62305.1 | CotA [Bacillus subtilis] spore copper-dependent | 91 | 94 | L . . . RYV |
| | laccase [Bacillus atrophaeus 1942] | | | |
| YP_003972023.1 | >gb\|ADP31092.1\|spore copper-dependent | 82 | 91 | L . . . RYV |
| | laccase (outer coat) [Bacillus atrophaeus 1942] | | | |
| | spore copper-dependent laccase [Bacillus | | | |
| | amyloliquefaciens DSM 7] >emb\|CBI41748.1\| | | | |
| | spore copper-dependent laccase [Bacillus | | | |
| | amyloliquefaciens DSM 7] >gb\|AEB22768.1\| | | | |
| | spore | | | |
| YP_003919218.1 | copper-dependent laccase [Bacillus | 77 | 89 | L . . . RYV |
| | amyloliquefaciens TA208] >gb\|AEB62213.1\| | | | |
| | spore copper-dependent laccase [Bacillus | | | |
| | amyloliquefaciens LL3] >gb\|AEK87755.1\|spore | | | |
| | copper-dependent laccase [Bacillus | | | |
| | amyloliquefaciens XH7] | | | |
| YP_001420286.1 | CotA [Bacillus amyloliquefaciens FZB42] | 77 | 89 | L . . . RYV |
| | >gb\|ABS73055.1\|CotA Bacillus | | | |
| | amyloliquefaciens FZB42] spore coat protein A | | | |
| | [Bacillus pumilus ATCC 7061] | | | |
| ZP_03054403.1 | >gb\|EDW21710.1\|spore coat protein A [Bacillus | 69 | 79 | L . . . RYV |
| | pumilus ATCC 7061] | | | |
| YP_001485796.1 | outer spore coat protein A [Bacillus pumilus | 68 | 79 | L . . . RYV |
| | SAFR-032] >gb\|ABV61236.1\|outer spore coat | | | |
| | protein A [Bacillus pumilus SAFR-032] | | | |
| ZP_08001338.1 | CotA protein [Bacillus sp. BT1B_CT2] | 65 | 77 | L . . . RYV |
| | >gb\|EFV71562.1\|CotA protein [Bacillus sp. | | | |
| | BT1B_CT2] | | | |
| YP_077905.1 | spore coat protein [Bacillus licheniformis ATCC | 65 | 77 | L . . . RYV |
| | 14580] >ref\|YP_090310.1\|CotA [Bacillus | | | |
| | licheniformis ATCC 14580] >gb\|AAU22267.1\| | | | |
| | spore coat protein (outer) [Bacillus licheniformis | | | |
| | ATCC 14580] >gb\|AAU39617.1\|CotA [Bacillus | | | |
| | licheniformis ATCC 14580] | | | |
| NP_692267.1 | spore outer coat protein [Oceanobacillus | 60 | 74 | L . . . DYV |
| | iheyensis HTE831] >dbj\|BAC13302.1\|spore | | | |
| | coat protein (outer) [Oceanobacillus iheyensis | | | |
| | HTE831] | | | |
| YP_176145.1 | spore coat protein [Bacillus clausii KSM-K16] | 59 | 75 | L . . . YYV |
| | >dbj\|BAD65184.1\|spore coat protein [Bacillus | | | |
| | clausii KSM-K16] | | | |
| ZP_04432136.1 | Bilirubin oxidase [Bacillus coagulans 36D1] | 60 | 74 | L . . . DYV |
| | >gb\|EEN93171.1\|Bilirubin oxidase [Bacillus | | | |
| | coagulans 36D1] | | | |
| YP_004569824.1 | Bilirubin oxidase [Bacillus coagulans 2-6] | 59 | 73 | L . . . DYV |
| | >gb\|AEH54438.1\|Bilirubin oxidase [Bacillus | | | |
| | coagulans 2-6] | | | |
| ZP_04217826.1 | Multicopper oxidase, type 2 [Bacillus cereus | 52 | 71 | L . . . DYV |
| | Rock3-44] >gb\|EEL50489.1\|Multicopper | | | |
| | oxidase, type 2 [Bacillus cereus Rock3-44] | | | |
| ZP_04295322.1 | Multicopper oxidase, type 2 [Bacillus cereus | 53 | 70 | L . . . DYV |
| | AH621] >gb\|EEK73233.1\|Multicopper oxidase, | | | |
| | type 2 [Bacillus cereus AH621] | | | |
| ZP_04201013.1 | Spore coat protein A [Bacillus cereus AH603] | 53 | 70 | L . . . DYV |
| | >gb\|EEL67287.1\|Spore coat protein A [Bacillus | | | |
| | cereus AH603] | | | |
| ZP_04180582.1 | Spore coat protein A [Bacillus cereus AH1272] | 53 | 70 | L . . . DYV |
| | >gb\|EEL87731.1\|Spore coat protein A [Bacillus | | | |
| | cereus AH1272] | | | |
| ZP_04150084.1 | Multicopper oxidase, type 2 [Bacillus | 51 | 67 | L . . . TYP |
| | pseudomycoides DSM 12442] | | | |
| | >gb\|EEM18231.1\|Multicopper oxidase, type 2 | | | |
| | [Bacillus pseudomycoides DSM 12442] | | | |
| YP_003639715.1 | Bilirubin oxidase [Thermincola sp. JR] | 53 | 68 | L . . . VFP |
| | >gb\|ADG81814.1\|Bilirubin oxidase | | | |
| | [Thermincola potens JR] | | | |
| ZP_04155855.1 | Multicopper oxidase, type 2 [Bacillus mycoides | 52 | 67 | L . . . TYP |
| | Rock3-17] >g b\|EEM12426.1\|Multicopper | | | |
| | oxidase, type 2 [Bacillus mycoides Rock3-17] | | | |
| ZP_04161675.1 | Multicopper oxidase, type 2 [Bacillus mycoides | 52 | 67 | L . . . TYP |
| | Rock1-4] >gb\|EEM06612.1\|Multicopper | | | |
| | oxidase, type 2 [Bacillus mycoides Rock1-4] | | | |
| ZP_08642538.1 | spore coat protein A [Brevibacillus laterosporus | 51 | 68 | L . . . TYM |
| | LMG 15441] >gb\|EGP32769.1\|spore coat | | | |
| | protein A [Brevibacillus laterosporus LMG | | | |
| | 15441] | | | |
| ZP_08679639.1 | spore coat protein A [Sporosarcina | 50 | 65 | L . . . RYV |
| | newyorkensis 2681] >gb\|EGQ24147.1\|spore | | | |
| | coat protein A [Sporosarcina newyorkensis | | | |
| | 2681] | | | |
| YP_001697777.1 | spore coat protein A [Lysinibacillus sphaericus | 52 | 67 | L . . . NYM |
| | C3-41] >gb\|ACA39647.1\|Spore coat protein A | | | |
| | [Lysinibacillus sphaericus C3-41] | | | |
| ZP_05132033.1 | spore coat protein [Clostridium sp. 7_2_43FAA] | 51 | 65 | L . . . NYV |
| | >gb\|EEH98927.1\|spore coat protein | | | |
| | [Clostridium sp.7_2_43FAA] | | | |
| ZP_01723401.1 | spore coat protein (outer) [Bacillus sp. B14905] | 52 | 66 | L . . . NYM |
| | >gb\|EAZ86095.1\|spore coat protein (outer) | | | |
| | [Bacillus sp. B14905] | | | |
| ZP_07051936.1 | spore coat protein A [Lysinibacillus fusiformis | 50 | 66 | L . . . NYM |
| | ZC1]>gb\|EFI66832.1\|spore coat protein A | | | |
| | [Lysinibacillus fusiformis ZC1] | | | |

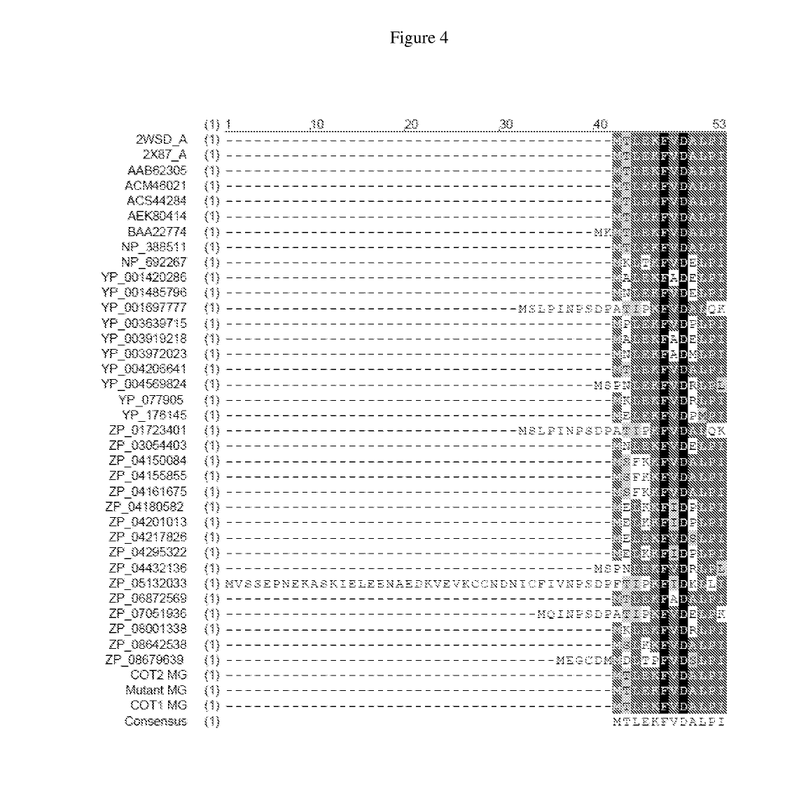

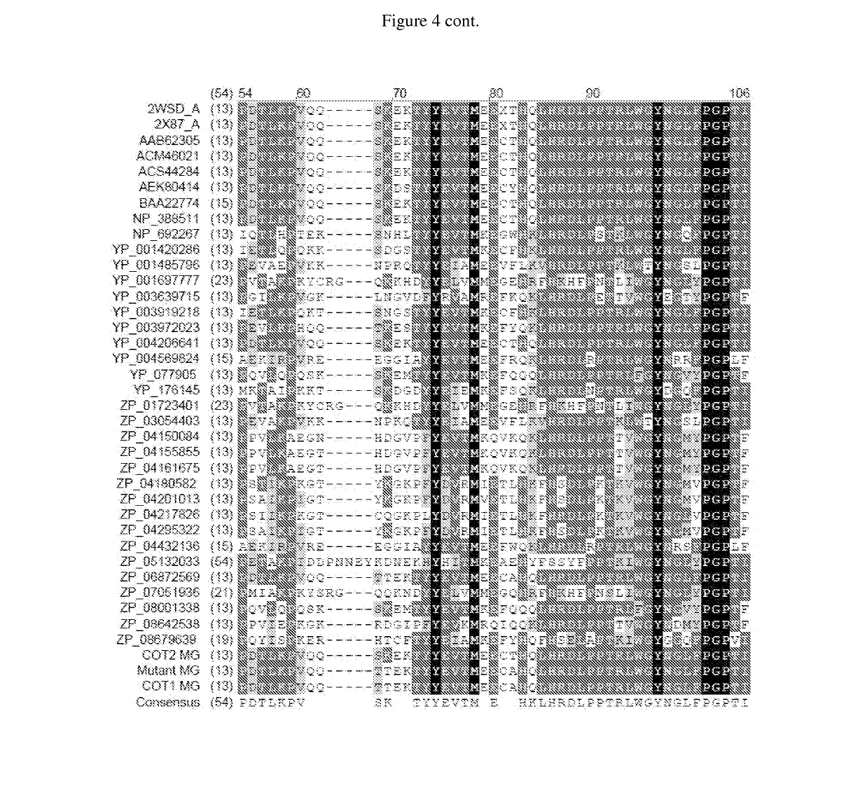

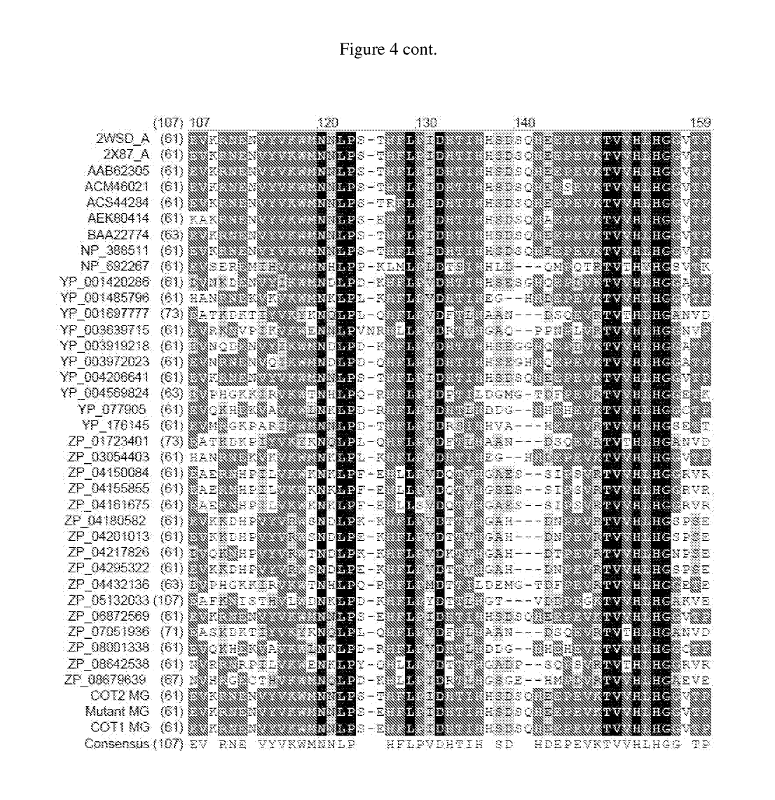

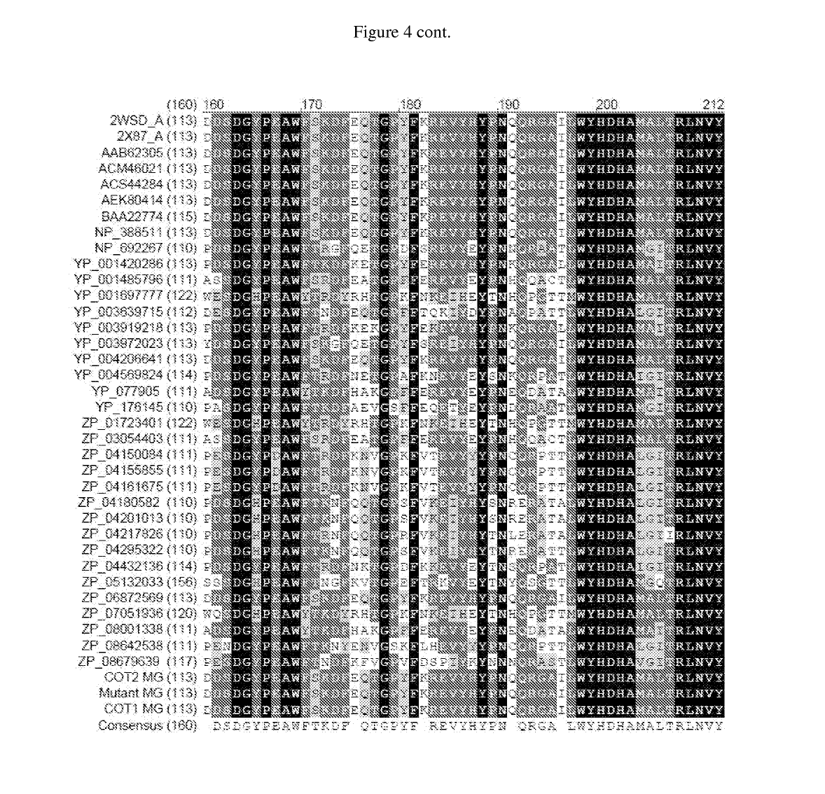

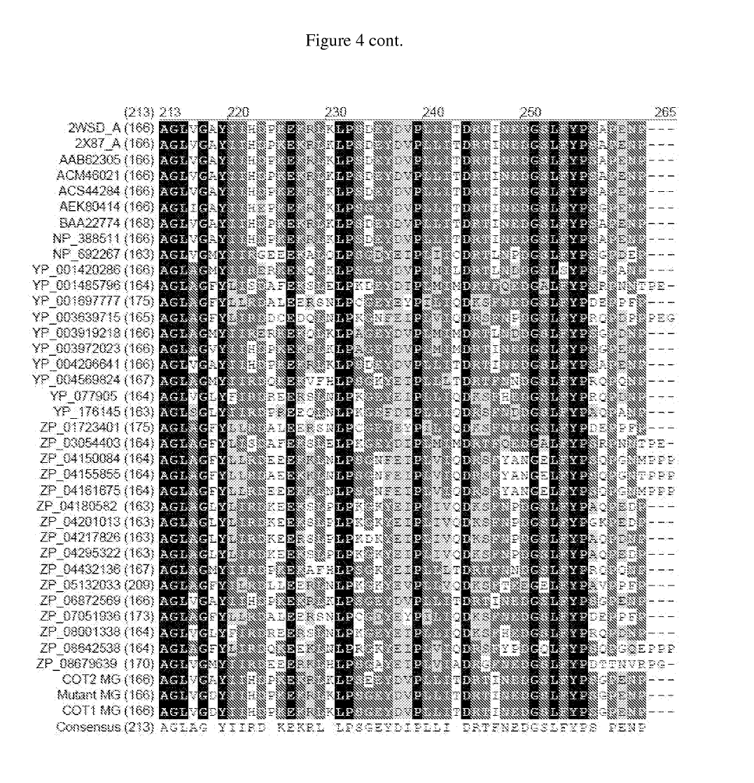

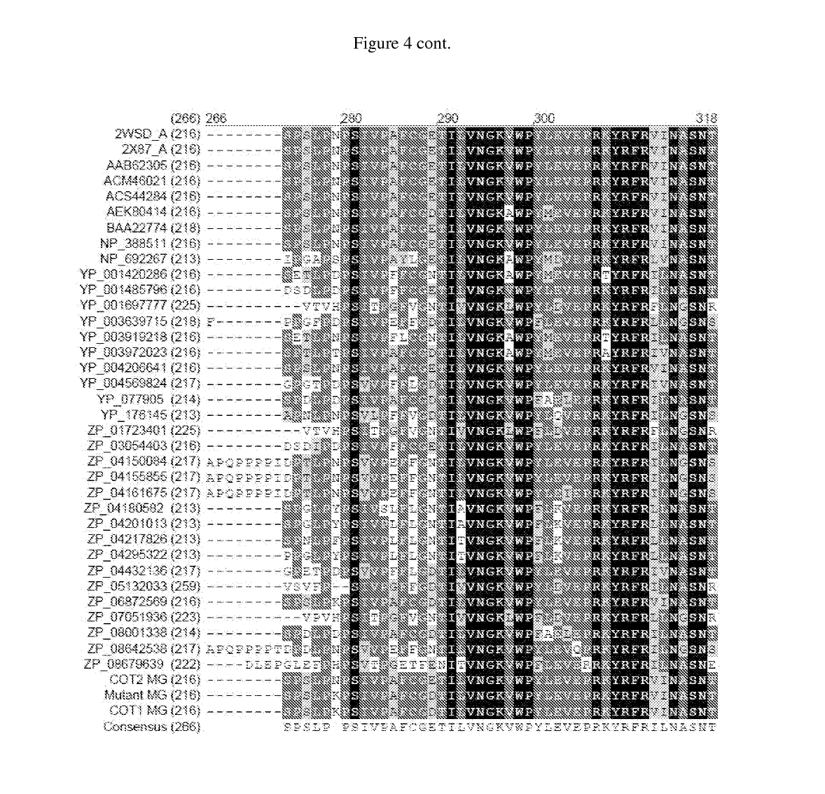

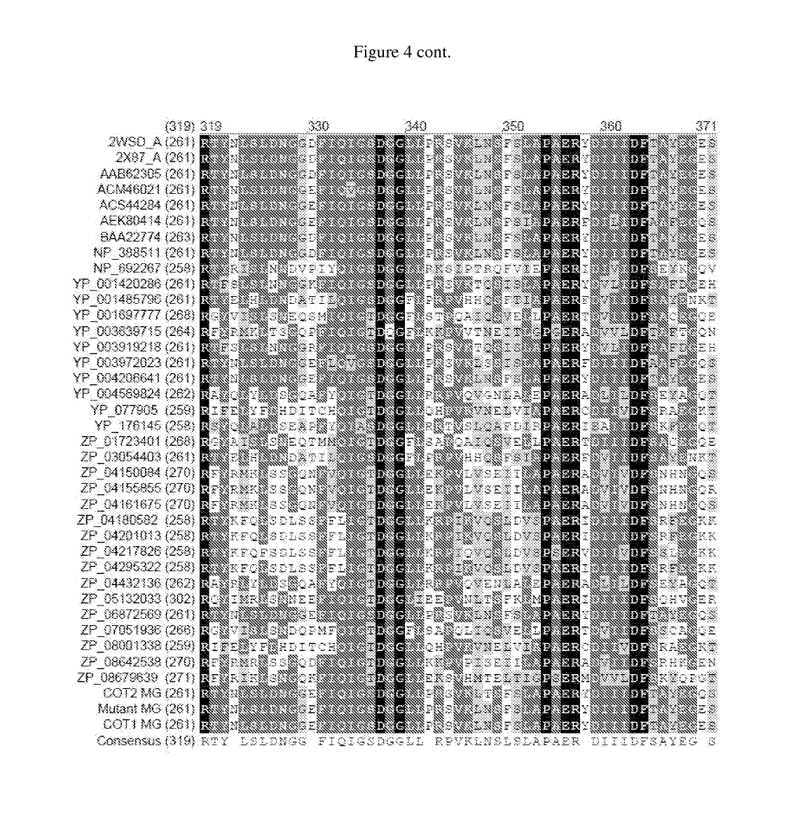

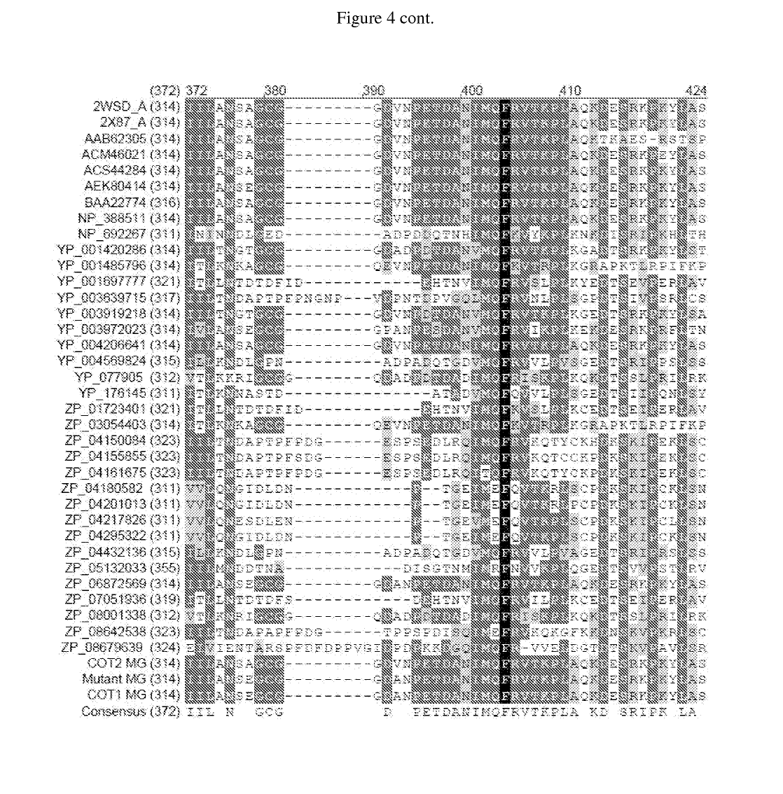

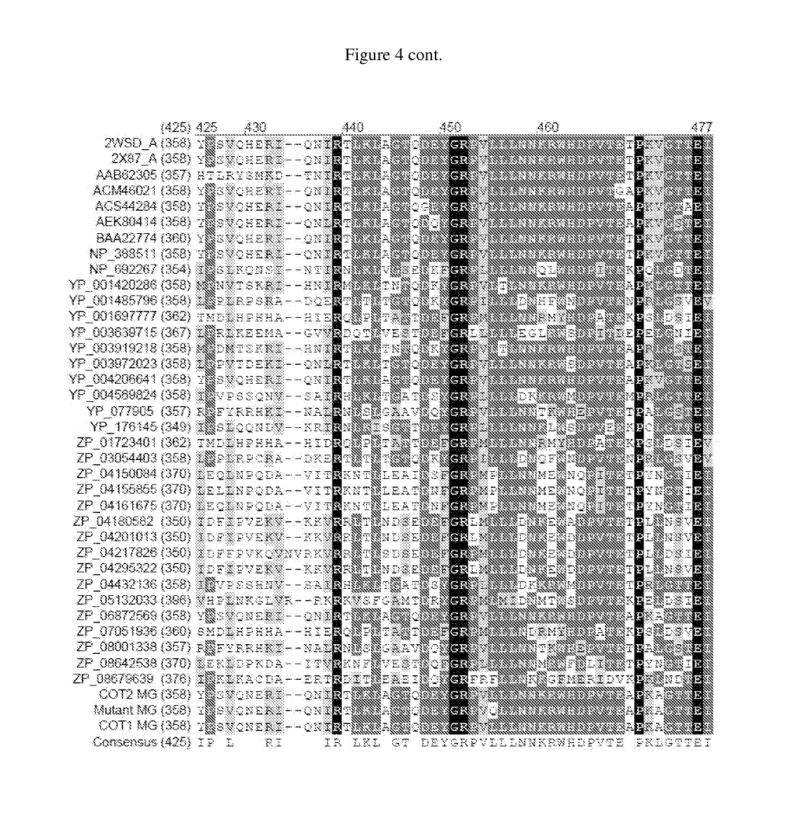

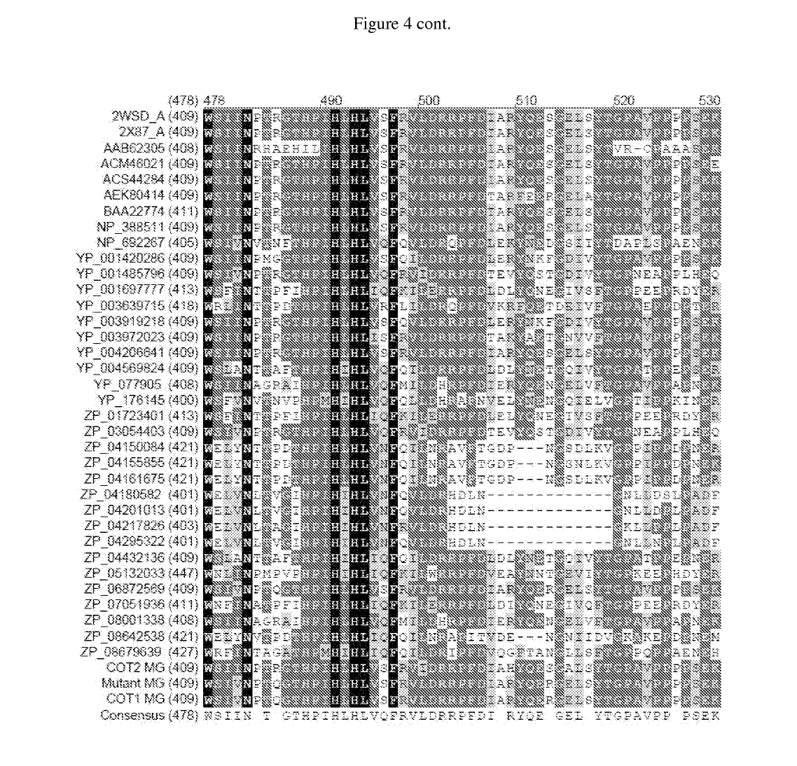

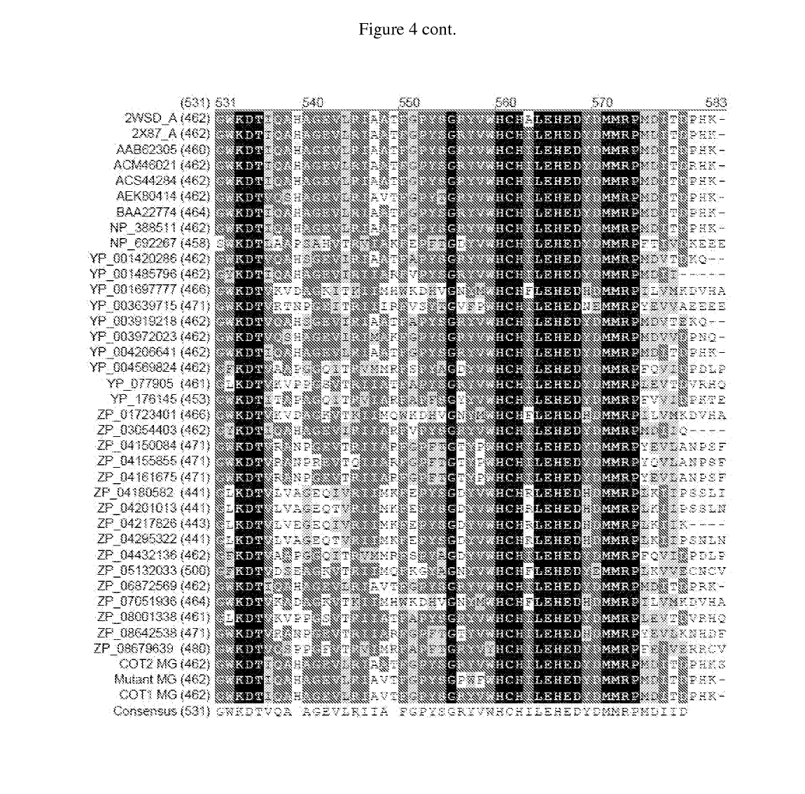

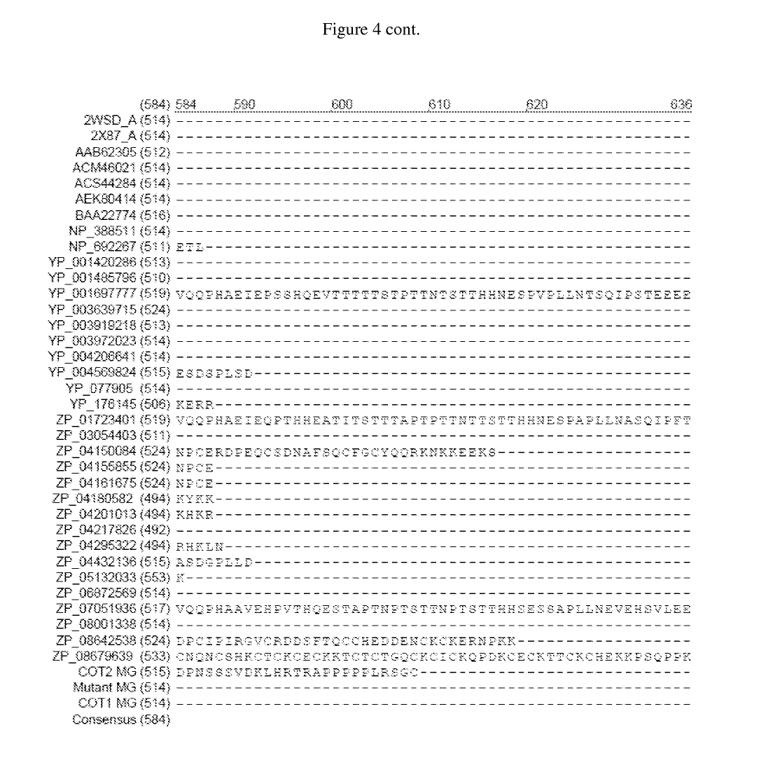

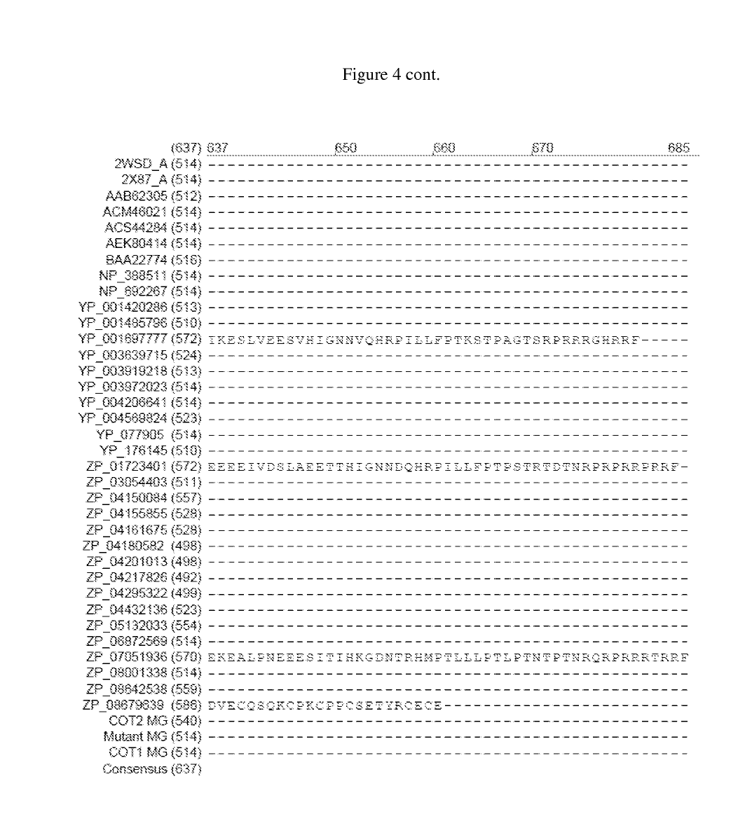

In order to create a more general picture of the structure of the related sequences, multiple alignments of the revealed sequences were performed. Over 30 most similar sequences ranging from 98 to 50% identity to the query sequences were downloaded to VectorNTI® software (Invitrogen) and arranged in a multiple alignment in the same order as in the BLAST list (FIG. 4). The alignment confirmed the uniqueness of the present amino acid substitutions.

Mutations corresponding to the Q386 mutation and/or P487/W488/F489 triple mutation shown in SEQ ID NO:3 may be introduced to any of the amino acid sequences disclosed herein, or other homologous sequences, by standard methods known in the art, such as site-directed mutagenesis, in order to improve their laccase activity in alkaline conditions. Kits for performing site-directed mutagenesis are commercially available in the art (e.g. QuikChange® II XL Site-Directed Mutagenesis kit by Agilent Technologies). Further suitable methods for introducing the above mutations into a recombinant gene are disclosed e.g. in Methods in Molecular Biology, Vol 182, “In vitro mutagenesis protocols”, Eds Jeff Braman, Humana Press 2002). Thus, some embodiments of the present invention relate to laccase variants or mutants which comprise Glutamine (Q) in a position which corresponds to the position 386 of the amino acid sequence depicted in SEQ ID NO:3 (denoted as MUT1) and/or Proline-Tryptophan-Phenylalanine (PWF) triple mutation in a position which corresponds to the position 487-489 of the amino acid sequence depicted in SEQ ID NO:3 (MUT2), and have an increased laccase activity in alkaline conditions as compared to that of a corresponding non-mutated control variant (Table 2).

TABLE 2
Effect of mutations MUT1 and the triple mutation (MUT2) on
relative activities of laccase proteins at different pH. All mutations
were beneficial for activity even at acidic pH; however a much
larger effect was observed at elevated pH values.

| | % act at pH 5 | % act at pH 7 | % act at pH 9 |
|---|---|---|---|
| SEQ ID NO: 1 | 100 | 60 | 23 |
| SEQ ID NO: 1 + | 140 | 170 | 220 |
| MUT1 | | | |
| SEQ ID NO: 1 + | 120 | 130 | 150 |
| MUT2 | | | |
| SEQ ID NO: 1 + | 180 | 210 | 300 |
| MUT1 + | | | |
| MUT2 | | | |
| SEQ ID NO: 2 | 100 | 60 | 25 |
| SEQ ID NO: 2 + | 130 | 160 | 200 |
| MUT1 | | | |
| SEQ ID NO: 2 + | 120 | 130 | 150 |
| MUT2I | | | |
| SEQ ID NO: 2 + | 160 | 200 | 300 |
| MUT1 + | | | |
| MUT2 | | | |

Amino acid sequences revealed in the Blast search may be represented as a consensus sequence. SEQ ID NO:6 represent a consensus sequence of 33 amino acid sequences most closely related to the COT1 and COT2 query sequences. Thus, some embodiments of the present invention relate to laccase variants comprising an amino acid sequence depicted in SEQ ID NO:6 introduced with a MUT1 and/or MUT2 mutation.

In some other embodiments, the present laccase variants, i.e. homologues, having an increased enzyme activity in alkaline conditions comprise an amino acid sequence which has at least 50% sequence identity with the variants comprising an amino acid sequence selected from the group consisting of SEQ ID NO:3 (comprising MUT1+MUT2); SEQ ID NO:4 (comprising MUT1), and SEQ ID NO:5 (comprising MUT2). In other embodiments, said amino acid sequence is selected from a group consisting of SEQ ID NO:1, SEQ ID NO:2, SEQ ID NO:6, and any of the sequences shown FIG. 4, further comprising a mutation corresponding to MUT1 and/or MUT2. In further embodiments, the present invention relates to laccase variants which comprise an amino acid sequence having a degree of identity to any of the above-mentioned reference sequences of at least about 55%, preferably about 65%, more preferably about 75%, still more preferably about 85%, and even more preferably about 95%, 96%, 97%, 98%, or 99%, and which retain increased laccase activity in alkaline conditions. In some embodiments, the degree of identity corresponds to any value between the above-mentioned integers.

As used herein, the degree of identity between two or more amino acid sequences is equivalent to a function of the number of identical positions shared by the sequences (i.e., % identity=# of identical positions/total # of positions×100), excluding gaps, which need to be introduced for optimal alignment of the two sequences, and overhangs. The comparison of sequences and determination of percent identity between two or more sequences can be accomplished using standard methods known in the art.

The present laccase variants may comprise conservative amino acid substitutions as compared to any of the sequences depicted in SEQ ID NO:1, SEQ ID NO:2, SEQ ID NO:3, SEQ ID NO:4, SEQ ID NO:5, SEQ ID NO:6, and FIG. 4. The term “conservative amino acid substitution” refers to a replacement of an amino acid with a similar amino acid as known in the art. Conservative amino acid substitutions do not significantly affect the folding and/or activity of a protein sequence variant. Typical non-limiting examples of such conservative amino acid substitutions include substitution of glutamate for aspartate or vice versa.

The present laccase variants may further comprise amino acid deletions and/or additions as long as they retain their increased laccase activity in alkaline conditions. In this context, the term “functional fragment” refers to a truncated laccase polypeptide retaining said increased enzyme activity in alkaline conditions.

As used herein, the term “conservative variant” refers to polypeptides comprising conservative amino acid substitutions, deletions and/or additions, and retaining their enzymatic properties, especially increased laccase activity in alkaline conditions.

The present laccase polypeptides or proteins may be fused to additional sequences, by attaching or inserting, including, but not limited to, affinity tags, facilitating protein purification (S-tag, maltose binding domain, chtin binding domain), domains or sequences assisting folding (such as thioredoxin domain, SUMO protein), sequences affecting protein localization (periplasmic localization signals etc), proteins bearing additional function, such as green fluorescent protein (GFP), or sequences representing another enzymatic activity. Other suitable fusion partners for the present laccases are known to those skilled in the art.

The present invention also relates to isolated polynucleotides encoding any of the laccase variants disclosed herein. Means and methods for cloning and isolating such polynucleotides are well known in the art.

Furthermore, the present invention relates to vectors comprising the present polynucleotides operably linked to one or more control sequences. Suitable control sequences are readily available in the art and include, but are not limited to, promoter, leader, polyadenylation, and signal sequences.

Laccase variants according to various embodiments of the present invention may be obtained by standard recombinant methods known in the art. Briefly, such a method may comprise the steps of i) culturing a desired recombinant host cell under conditions suitable for the production of a present laccase polypeptide variant, and ii) recovering the polypeptide variant obtained. A large number of vector-host systems known in the art may be used for recombinant production of laccase variants. Possible vectors include, but are not limited to, plasmids or modified viruses which are maintained in the host cell as autonomous DNA molecule or integrated in genomic DNA. The vector system must be compatible with the host cell used as well known in the art. Non-limiting examples of suitable host cells include bacteria (e.g. E. coli, bacilli), yeast (e.g. Pichia Pastoris, Saccharomyces Cerevisae), fungi (e.g. filamentous fungi) insect cells (e.g. Sf9).

Recovery of a laccase variant produced by a host cell may be performed by any technique known to those skilled in the art. Possible techniques include, but are not limited to secretion of the protein into the expression medium, and purification of the protein from cellular biomass.

The production method may further comprise a step of purifying the laccase variant obtained. For thermostable laccases, non-limiting examples of such methods include heating of the disintegrated cells and removing coagulated thermo labile proteins from the solution. For secreted proteins, non-limiting examples of such methods include ion exchange chromatography, and ultra-filtration of the expression medium. It is important that the purification method of choice is such that the purified protein retains its laccase activity.

The present laccase variants may be used in a wide range of different industrial processes and applications, such as in pulp delignification, textile dye bleaching, wastewater detoxifixation, xenobiotic detoxification, and detergent manufacturing. The increased operable pH range of the disclosed laccase variants makes them particularly suitable for industrial waste water treatment processes.

It will be obvious to a person skilled in the art that, as the technology advances, the inventive concept can be implemented in various ways. The invention and its embodiments are not limited to the examples described below but may vary within the scope of the claims.

### Example 1

Construction of laccase variants bearing MUT1

Mutations according to the present invention were introduced into various recombinant genes by standard site-directed mutagenesis. For instance, MUT1 (L386Q substitution) was introduced into the gene of Multicopper oxidase, type 2 from Bacillus pseudomycoides (ZP_{—}04150084), which has approximately 50% sequence identity to the COT1 (SEQ ID NO:1) and COT2 (SEQ ID NO.2) laccases, by PCR amplifying the coding sequence of this gene (accession number NZ_ACMX01000022) from genomic DNA of Bacillus pseudomycoides and cloning it into a pET20 plasmid vector.

To this end, two series of separate PCR reactions were carried out: (1) with Primer1 (5′-CGCCGTCTCACATGTCTTTTAAAAAATTTGTC-GATGCATTACC-3′; SEQ ID NO: 7) and Primer4 (5′-ATAGTT-TTGGACGCCCTATGCCATTATTAAATAACATGG-AGT-3′; SEQ ID NO: 8), and (2) with Primer2 (5′-CGCGGATCCGATGATTTCTCTTCTTTTTTATTTTT-CCGTTG-3′; SEQ ID NO:9) and Primer3 (5′-ACTCCA-TGTTATTTAATAA-TGGCATAGGGCGTCCAAAACTAT-3′; SEQ ID NO.10).

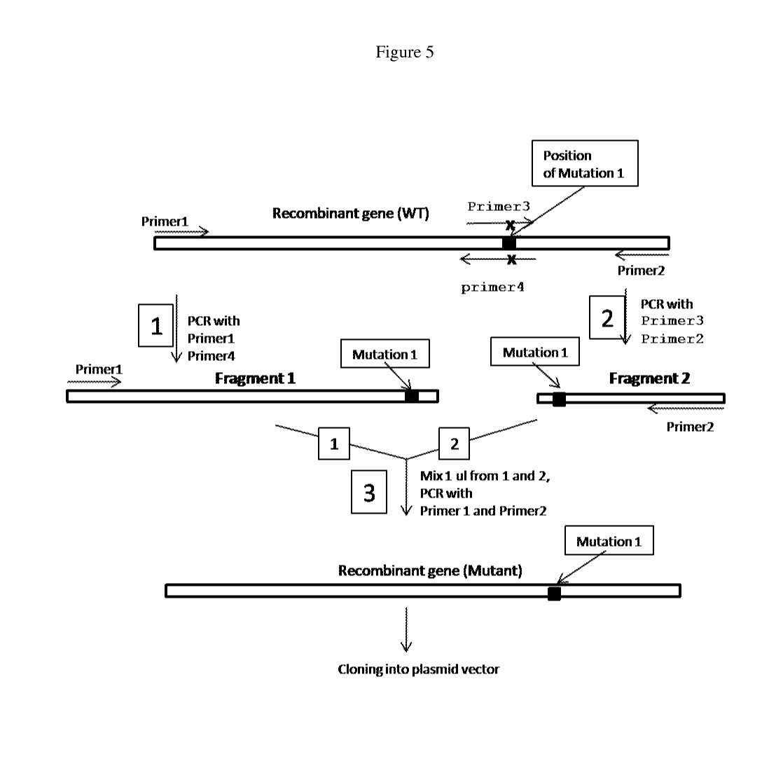

In both PCR series, recombinant wild type gene was used as the template. Aliquots of 1 μl from reactions (1) and (2) were combined and used as template for PCR reaction with Primer 1 and Primer 2 (see above). Product of this reaction containing the mutant sequence of the gene was cloned in a plasmid vector for expression in E. coli. Schematic representation of this mutagenesis strategy is presented in FIG. 5.

### Example 2

Relative activity measurement of laccase variants at different pH using 2,6-DMP

In the present experiments, 2,6-Dimethoxyphenol (2,6-DMP), which can be oxidized by wild type COT1 and COT2 laccases readily at pH 5 but much more slowly at pH 9, was chosen as the substrate.

Two forms of each enzyme—one possessing the mutation (Mut) and one without the mutation (further called wild type, WT) was tested in 2,6-Dimethoxyphenol (2,6-DMP) oxidation reactions at various pH. Reaction course was monitored by Absorbance at 500 nM.

Initial rates of the reactions were measured in OD(500)/min. Initial rate (V) is velocity of the reaction in the time range when the colour develops linearly with time. Similar reactions were carried out at different substrate (2,6-DMP) concentrations (see protocol below). Then maximum initial rate (Vmax) was determined at each pH (this rate was observed at saturating substrate concentrations).

In order to determine relative alkaline activity, for each enzyme its Vmax at pH5 was taken for 100%, and relative activity at pH7 or pH9 was determined as a fraction of this activity.

As an example, 2,6-DMP concentration of 0.5 mM was saturating for both WT and MUT enzymes at pH5 through pH9. MOPS buffer (3-(N-Morpholino) propane sulfonic acid, Sigma) was used as a reaction medium. Vmax of these two enzymes were determined according to the protocol:

| | |
|---|---|
| MOPS 100 mM pH (5-9) | 90 μl, |
| 2,6-DMP 5 mM | 10 μl, |
| Laccase (WT or MUT) | 2 μl, |

Incubation 10 min at 60 C.
Absorbance at 500 nm was measured by titer plate reader.

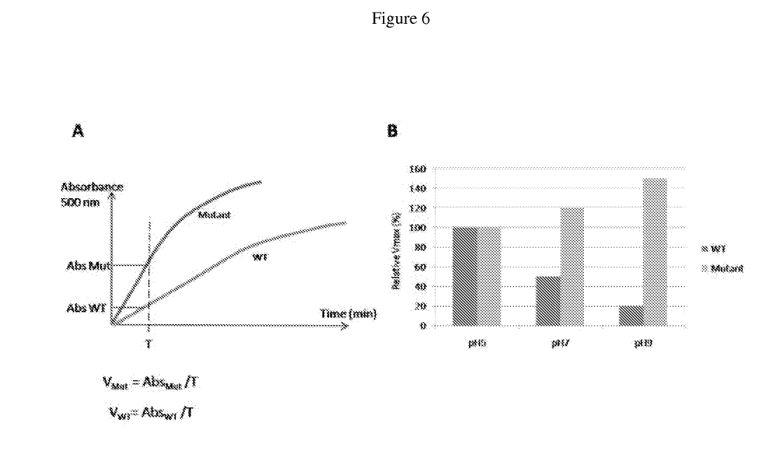

As demonstrated in FIG. 6, introducing MUT1 into the laccase polypeptide increases its relative activity at pH9 approximately 7-fold as compared to the non-mutated enzyme.

As well known to a person skilled in the art, the relative laccase activity at different pHs may be measured by any other substrate suitable for the laccase variant in question as long as the other substrate cab be oxidized at the same pH range (preferably pH 5 to pH 9). Also other parameters such as temperature may be adjusted to the particular laccase variant in question.

1. An enzyme having laccase activity, wherein the enzyme comprises a glutamine residue situated within 6Å of a type 1 Copper ion in the 3-dimentional structure of the enzyme.

2. The enzyme of claim 1, wherein the enzyme comprises an amino acid sequence having at least 50% identity to an amino acid sequence selected from the group consisting of SEQ ID NO:3, SEQ ID NO:4, and SEQ ID NO:5; and
wherein the enzyme comprises at least one amino acid variant selected from the group consisting of a glutamine (Q) in a position which corresponds to position 386 of SEQ ID NO:3 and a Proline- Tryptophan-Phenylalanine (PWF) sequence in a position which corresponds to positions 487-489 of SEQ ID NO:3.

3. The enzyme of claim 2, wherein the enzyme has increased laccase activity in alkaline conditions as compared to that of a corresponding control enzyme lacking the at least one amino acid variant.

4. The enzyme of claim 1, wherein the enzyme comprises an amino acid sequence selected from the group consisting of SEQ ID NO:1 , SEQ ID NO:2, SEQ ID NO:6, SEQ ID NOs:11-48, and conservative variants and functional fragments thereof, introduced with at least one mutation selected from the group consisting of a Q386 mutation and a P487/W488/F489 triple mutation depicted in SEQ ID NO:3.

5. A nucleic acid molecule encoding the enzyme of claim 1.

6. A vector comprising the nucleic acid molecule of claim 5.

7. A recombinant host cell comprising the vector of claim 6.

8. A method of producing an enzyme, the method comprising:
producing the enzyme in the recombinant host cell of claim 6, and
recovering the enzyme from the host cell.

9. The method according to claim 8, further comprising purifying the enzyme.

10. A method of pulp delignification, the method comprising treating a pulp comprising lignin with the enzyme of claim 1.

11. A method of textile dye bleaching, the method comprising treating a textile with the enzyme of claim 1.

12. A method of wastewater detoxifixation, the method comprising treating wastewater with the enzyme of claim 1.

13. A method of xenobiotic detoxification, the method comprising treating the xenobiotic with the enzyme of claim 1.
